(19) United States
(12) Patent Application Publication
(10) Pub. No.: US 2024/0255507 A1
(43) Pub. Date: Aug. 1, 2024

(54) METHOD FOR DETECTING ANTI-SARS-COV-2 ANTIBODY, CHIMERIC PROTEIN, POLYNUCLEOTIDE, EXPRESSION VECTOR, HOST CELL, METHOD FOR PRODUCING CHIMERIC PROTEIN, AND REAGENT

(71) Applicant: Sysmex Corporation, Kobe-shi, Hyogo (JP)

(72) Inventors: Yusuke ATARASHI, Kobe-shi, Hyogo (JP); Jeeeun KIM, Kobe-shi, Hyogo (JP); Masatoshi SUGANUMA, Kobe-shi, Hyogo (JP); Nobuyuki IDE, Kobe-shi, Hyogo (JP); Kenta NODA, Kobe-shi, Hyogo (JP)

(73) Assignee: Sysmex Corporation, Kobe-shi, Hyogo (JP)

(21) Appl. No.: 18/508,791

(22) Filed: Nov. 14, 2023

(30) Foreign Application Priority Data
Nov. 16, 2022 (JP) 2022-183369

(51) Int. Cl. G01N 33/569; C07K 14/005

(52) U.S. Cl. CPC G01N 33/56983; C07K 14/005; C12N 2770/20022; G01N 2333/165; G01N 2469/20

(57) ABSTRACT

Disclosed is a method for detecting an anti-SARS-CoV-2 antibody, the method comprising: detecting an antibody against a chimeric protein comprising an amino acid sequence of a receptor binding region of SARS-CoV-2 and an amino acid sequence of a receptor binding region of a coronavirus belonging to Nobecovirus subgenus in a specimen obtained from a subject using the chimeric protein, the chimeric protein being a protein represented by (i) a protein comprising an amino acid sequence set forth in SEQ ID NO: 1 or 2; or (ii) a protein comprising an amino acid sequence having 80% or more of identity to the amino acid sequence set forth in SEQ ID NO: 1 or 2 and binding to an antibody that recognizes the receptor binding region of SARS-CoV-2.

### CROSS REFERENCE TO RELATED APPLICATIONS

This application claims priority from prior Japanese Patent Application No. 2022-183369, filed on Nov. 16, 2022, entitled “Method for detecting anti-SARS-CoV-2 antibody, chimeric protein, polynucleotide, expression vector, host cell, method for producing chimeric protein, and reagent”, the entire contents of which are incorporated herein by reference.

### INCORPORATION BY REFERENCE OF SEQUENCE LISTING

The content of the electronically submitted sequence listing, file name: Q291879_Sequence listing as filed.xml; size: 49,152 bytes; and date of creation: Nov. 13, 2023, filed herewith, is incorporated herein by reference in its entirety.

### FIELD OF THE INVENTION

The present invention relates to a method for detecting an anti-SARS-CoV-2 antibody. The present invention relates to a chimeric protein. The present invention relates to a polynucleotide encoding a chimeric protein. The present invention relates to an expression vector including a polynucleotide. The present invention relates to a host cell into which a polynucleotide or an expression vector is introduced. The present invention relates to a method for producing a chimeric protein. The present invention relates to a reagent for detecting an anti-SARS-CoV-2 antibody.

### BACKGROUND

Severe acute respiratory syndrome coronavirus 2 (SARS-CoV-2) recognizes angiotensin converting enzyme 2 (ACE2) on the surface of cells of a living body as a receptor, and infects the cells. More specifically, a spike protein on the surface of the SARS-CoV-2 binds to ACE2, whereby the SARS-CoV-2 fuses with the cell membrane and enters the cell. The spike protein includes a receptor binding region (RBD) that is a binding site with ACE2. The RBD also includes a receptor binding motif (RBM) that mediates contact with ACE2 and a core that constitutes a region other than the RBM.

For detection and analysis of an antibody against SARS-CoV-2, a recombinant protein of SARS-CoV-2's RBD is used. For example, Starr TN. et al., SARS-CoV-2 RBD antibodies that maximize breadth and resistance to escape, Nature, vol. 597, pp. 97-102, 2021 describes that various antibodies against the RBD of SARS-CoV-2 were comprehensively examined using a recombinant protein of RBD. In addition, Shang J. et al., Structural basis of receptor recognition by SARS-CoV-2, Nature, vol. 581, pp. 221-224, 2020 describes that a chimeric RBD including an RBM of SARS-CoV-2 and a core of SARS-CoV-1 was used in order to examine a mechanism in which SARS-CoV-2 recognizes ACE2 as a receptor.

### SUMMARY OF THE INVENTION

The scope of the present invention is defined solely by the appended claims, and is not affected to any degree by the statements within this summary.

A specimen of a SARS-CoV-2 infected person and a SARS-CoV-2 vaccinator is considered to include an antibody that binds to a part not involved in binding to ACE2 and an antibody that binds to a part involved in binding to ACE2. In the conventional SARS-CoV-2 detection, an antibody that binds to a part involved in binding to ACE2 could not be specifically measured. However, if such an antibody can be specifically measured, the ability to protect against infection with SARS-CoV-2 may be verified more accurately. An object of the present invention is to provide a means for detecting an anti-SARS-CoV-2 antibody that binds to a part involved in binding to ACE2.

The present inventors conceived the use of a chimeric protein in which a part not involved in binding to ACE2 in the RBD of SARS-CoV-2 is substituted with another coronavirus-derived RBD as an antigen for detecting an anti-SARS-CoV-2 antibody. Then, the present inventors have found that an anti-SARS-CoV-2 antibody can be detected from a specimen using a chimeric protein including an RBD of SARS-CoV-2 and an RBD of a coronavirus belonging to Nobecovirus subgenus, thereby completing the invention described in the following [1] to [14].

[1] A method for detecting an anti-SARS-CoV-2 antibody, the method comprising:
detecting an antibody against a chimeric protein comprising an amino acid sequence of an RBD of SARS-CoV-2 and an amino acid sequence of an RBD of a coronavirus belonging to Nobecovirus subgenus in a specimen obtained from a subject using the chimeric protein,
the chimeric protein being a protein represented by
(i) a protein comprising an amino acid sequence set forth in SEQ ID NO: 1 or 2; or
(ii) a protein comprising an amino acid sequence having 80% or more of identity to the amino acid sequence set forth in SEQ ID NO: 1 or 2 and binding to an antibody that recognizes the RBD of SARS-CoV-2.

[2] The detection method according to [1], wherein the protein (ii) includes an amino acid sequence having 90% or more of identity to the amino acid sequence set forth in SEQ ID NO: 1 or 2.

[3] The detection method according to [1], wherein the protein (ii) includes an amino acid sequence having 95% or more of identity to the amino acid sequence set forth in SEQ ID NO: 1 or 2.

[4] The detection method of according to any one of [1] to [3], wherein the detecting an antibody against the chimeric protein further includes: forming an immune complex of the chimeric protein, an antibody against the chimeric protein, and a capture body that binds to the antibody and has a labeling substance; and detecting a signal generated by the labeling substance included in the immune complex.

[5] The detection method according to [4], wherein a value based on the signal is an index of a presence of the antibody against the chimeric protein in the specimen.

[6] The detection method according to any one of [1] to [5], wherein the specimen is serum or plasma.

[7] A chimeric protein comprising:
an amino acid sequence of an RBD of SARS-CoV-2 and an amino acid sequence of an RBD of a coronavirus belonging to Nobecovirus subgenus, and being represented by
(i) a protein comprising an amino acid sequence set forth in SEQ ID NO: 1 or 2; or
(ii) a protein comprising an amino acid sequence having 80% or more of identity to the amino acid sequence set forth in SEQ ID NO: 1 or 2 and binding to an antibody that recognizes the RBD of SARS-CoV-2.

[8] The chimeric protein according to [7], wherein the protein (ii) includes an amino acid sequence having 90% or more of identity to the amino acid sequence set forth in SEQ ID NO: 1 or 2.

[9] The chimeric protein according to [7], wherein the protein (ii) includes an amino acid sequence having 95% or more of identity to the amino acid sequence set forth in SEQ ID NO: 1 or 2.

[10] A polynucleotide encoding the chimeric protein according to any one of [7] to [9].

[11] An expression vector including the polynucleotide according to [10].

[12] A host cell into which the polynucleotide according to or the expression vector according to is introduced.

[13] A method for producing a chimeric protein, including culturing the host cell according to and recovering the chimeric protein expressed by the host cell.

A reagent for detecting an anti-SARS-CoV-2 antibody, including the chimeric protein according to any one of [7] to [9].

According to the present invention, it is possible to provide a method for detecting an anti-SARS-CoV-2 antibody that binds to a part involved in binding to ACE2, a chimeric protein used in the method, a polynucleotide encoding the chimeric protein, an expression vector, a host cell, a method for producing a chimeric protein using the host cell, and a reagent for detecting an anti-SARS-CoV-2 antibody including the chimeric protein.

### BRIEF DESCRIPTION OF THE DRAWINGS

FIG. 1 is a diagram showing an example of a chimeric protein of the present embodiment; In the figure, a part surrounded by the solid line indicated by A indicates a region including the amino acid sequence of the RBD of SARS-CoV-2; The part surrounded by the broken line indicated by B indicates a region including the amino acid sequence of the RBD of the coronavirus belonging to Nobecovirus subgenus;

FIG. 2 is a diagram showing the amino acid sequence (SEQ ID NO: 4) of a region including the RBD in the amino acid sequence (SEQ ID NO: 3) of a spike protein of SARS-CoV-2; In the figure, a sequence denoted by a gray marker is an amino acid sequence of RBD; The underlined portion is an amino acid residue included in the first region described later; The portion surrounded by a square is an amino acid residue identified as a site where the RBD interacts with an antibody having neutralization activity;

FIG. 3 is a schematic view showing an example of a reagent kit for detecting an anti-SARS-CoV-2 antibody of the present embodiment; and

FIG. 4 is a schematic view showing an example of a reagent kit for detecting an anti-SARS-CoV-2 antibody of the present embodiment.

### DETAILED DESCRIPTION OF THE PREFERRED EMBODIMENTS

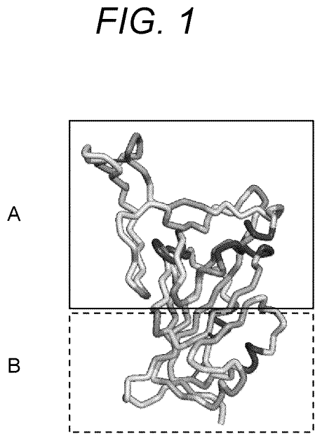

In the method for detecting an anti-SARS-CoV-2 antibody of the present embodiment (hereinafter, also referred to as a “detection method of the present embodiment”), an antibody against a chimeric protein in a specimen acquired from a subject is detected using a chimeric protein including the amino acid sequence of the RBD of SARS-CoV-2 and the amino acid sequence of the RBD of a coronavirus belonging to Nobecovirus subgenus. Hereinafter, this chimeric protein is also referred to as the “chimeric protein of the present embodiment”. Referring to FIG. 1, the chimeric protein of the present embodiment has a three-dimensional structure in which two of a part including the amino acid sequence of the RBD of SARS-CoV-2 (see A in the figure) and a part including the amino acid sequence of the RBD of a coronavirus belonging to Nobecovirus subgenus (see B in the figure) are fused. Specifically, the chimeric protein of the present embodiment is a protein shown in the following (i) or (ii).
(i) A protein including an amino acid sequence set forth in SEQ ID NO: 1 or 2;
(ii) A protein including an amino acid sequence having 80% or more of identity to the amino acid sequence set forth in SEQ ID NO. 1 or 2, and binding to an antibody that recognizes the RBD of SARS-CoV-2.

The amino acid sequences set forth in SEQ ID NOs: 1 and 2 are as follows.

(SEQ ID NO: 1)
RAQVAGFVRVTQRGSYCTPPYSVLQDPRPQPVVWRRYMLYDCVFD
FTVVVDSASFSTFKCYGVSPRRLASMCYGSVTLDVMRIRGDEVRQ
LFNRVTGKFSDYNYALPDNFYGCLHAWNSNNLDSKVGGNYNYLYR
LFRKSNLKPFERDISTEIYQAGSTPCNGVEGENCYFPLQSYGFQP
TNGVGYQPYRLAVITLKPAAGSKLVCPVANDTVVITDR
(SEQ ID NO: 2)
RAEAKKLVQVTQQEGSCAIPYTTILEPRPSPAAWVRATISNCTFD
FESLLRTASFSTFKCYGISPARLSTMCYAGVTLDIFKLRGDEVRQ
MLGSVTDKVSDYNYALPSNFYGCVHAWNSNNLDSKVGGNYNYLYR
LFRKSNLKPFERDISTEIYQAGSTPCNGVEGENCYFPLQSYGFQP
TNGVGYQPYRGVVVIGLTPASGSNLVCPKANDTHVIEGQ

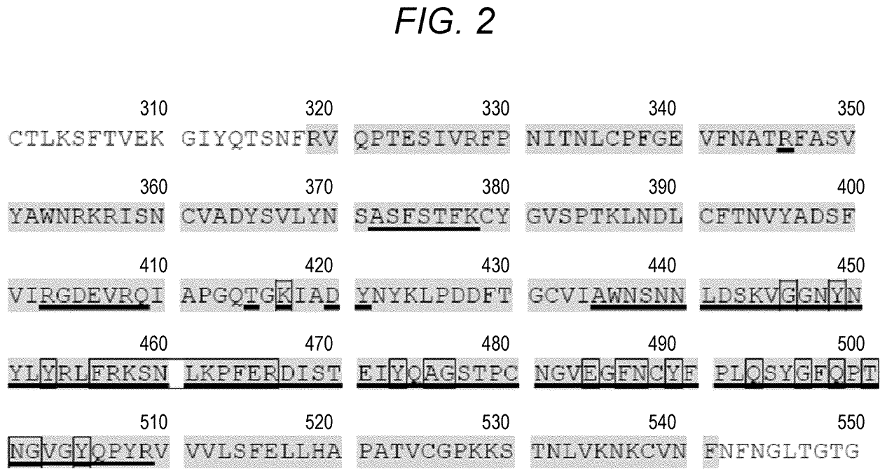

The amino acid sequences set forth in SEQ ID NOs: 1 and 2 include the amino acid sequences of a region involved in binding to ACE2 and a predetermined anti-SARS-CoV-2 antibody among the amino acid sequences of the RBD of SARS-CoV-2 (hereinafter, also referred to as a “first region”). In the amino acid sequences set forth in SEQ ID NOs: 1 and 2, the first region is an underlined amino acid sequence. The first region includes amino acid residues which interact with a predetermined anti-SARS-CoV-2 antibody (see FIG. 2). Therefore, the chimeric protein of the present embodiment can be used as an antigen for detecting an anti-SARS-CoV-2 antibody. When an antibody that binds to the chimeric protein of the present embodiment is included in a specimen collected from a subject, the antibody may be an anti-SARS-CoV-2 antibody. As described above, in the detection method of the present embodiment, an antibody against a chimeric protein included in a specimen acquired from a subject is detected as an anti-SARS-CoV-2 antibody.

The predetermined anti-SARS-CoV-2 antibody is a monoclonal antibody whose clone name is represented by CV30, REGNI10933, BD23, REGNI10987, and CR3022. The amino acid sequences of these antibodies are as shown in Tables 3 and 4 of Experimental Example 1 described later. It has been confirmed that these antibodies specifically bind to the RBD of SARS-CoV-2 and have an activity of inhibiting the binding between the RBD and ACE2 (hereinafter, also referred to as “neutralization activity”). That is, the five types of antibodies are neutralizing antibodies against SARS-CoV-2. In addition, the structure of these antibodies has already been analyzed in the cocrystal with the RBD, and these antibodies are registered in the Protein Data Bank (PDB), which is a known database. The chimeric protein of the present embodiment includes an amino acid residue involved in binding to the neutralizing antibody in the first region. In addition, it has been confirmed in Examples described later that the chimeric protein of the present embodiment can actually bind to the five types of antibodies. Therefore, when the specimen includes an antibody that binds to the chimeric protein of the present embodiment, the antibody can be an anti-SARS-CoV-2 antibody having a neutralization activity.

The first region is a region defined by the present inventors in the amino acid sequence of the RBD of SARS-CoV-2. Specifically, the first region is a region including amino acid residues identified based on the structural information of the cocrystal of ACE2 and the RBD and the cocrystal of a neutralizing antibody and the RBD. The amino acid residue identified in the amino acid sequence of the RBD of SARS-CoV-2 is a residue that interacts with ACE2 and neutralizing antibodies. The amino acid residue included in the first region was determined as follows.

First, based on the amino acid sequence and structure of the spike protein of SARS-CoV-2, the amino acid residues of the spike protein were numbered. The information referred to was obtained from known databases GenBank and Uniprot (GenBank Accession No. NC_045512.2, Uniprot ID: PODTC2). The RBD was defined based on the amino acid sequence and structure of the numbered spike proteins. Referring to FIG. 2, the amino acid sequence of the RBD of SARS-CoV-2 is an amino acid sequence (SEQ ID NO: 5) consisting of amino acid residues at positions 319 to 541 of the spike protein shown in SEQ ID NO: 3. Then, structural information of the cocrystal of ACE2 and a neutralizing antibody and the RBD of SARS-CoV-2 was obtained from PDB, and the amino acid residue that interacts with ACE2 and the neutralizing antibody was identified in the RBD. The referred information is data registered with a PDB ID: 6M0J. For example, in the amino acid sequence shown in FIG. 2, the amino acid residues that interact with the neutralizing antibody are residues surrounded by a square. The present inventors defined the first region based on the identified amino acid residues. That is, the first region is a region consisting of amino acid residues at positions 346, 372 to 378, 403 to 409, 415, 417, 420, 421, and 435 to 509 of the spike protein shown in SEQ ID NO: 3. These amino acid residues are underlined residues in the amino acid sequence shown in FIG. 2. As can be seen in FIG. 2, the first region includes amino acid residues that interact with the neutralizing antibody.

In the chimeric protein of the present embodiment, the site constituted by the amino acid sequence of the RBD of a coronavirus belonging to Nobecovirus subgenus is a region considered to be related to the maintenance of the three-dimensional structure as the RBD of the entire chimeric protein (hereinafter, also referred to as a “second region”). In the amino acid sequences set forth in SEQ ID NOs: 1 and 2, the second region is an amino acid sequence other than the underlined portion. It is considered that due to the presence of the second region, the first region also maintains the three-dimensional structure as an antigen recognized by the anti-SARS-CoV-2 antibody. In the amino acid sequence set forth in SEQ ID NO: 1, the second region includes an amino acid sequence derived from the RBD of the HKU9 strain of Nobecovirus subgenus. In the amino acid sequence set forth in SEQ ID NO: 2, the second region includes an amino acid sequence derived from the RBD of a GCCDC1 strain of Nobecovirus subgenus.

As shown in Experimental Example 1 described later, the second region was determined by sequence alignment by comparing the amino acid sequence of the RBD of a coronavirus belonging to Nobecovirus subgenus with the amino acid sequence of the RBD of SARS-CoV-2. Sequence alignment refers to comparably aligning a plurality of amino acid sequences. Specifically, first, an amino acid residue corresponding to the amino acid residue in the first region was identified in the amino acid sequence of the RBD of a coronavirus belonging to Nobecovirus subgenus. Then, the remaining sequence obtained by removing the specified amino acid residue from the amino acid sequence of the RBD of the coronavirus belonging to Nobecovirus subgenus was identified as the second region.

The second region has low (about 27%) identity to the amino acid sequence of the RBD of SARS-CoV-2. Therefore, even if the specimen includes an anti-SARS-CoV-2 antibody that does not bind to the first region, it is considered that the antibody does not bind to the second region of the chimeric protein of the present embodiment. Therefore, the anti-SARS-CoV-2 antibody that binds to the chimeric protein of the present embodiment can be an antibody that recognizes the first region.

The protein (ii) is also the chimeric protein of the present embodiment. That is, the chimeric protein of the present embodiment may contain an amino acid sequence having 80% or more, preferably 90% or more, more preferably 95% or more, and still more preferably 99% identity to the amino acid sequence set forth in SEQ ID NO: 1 or 2 as long as it binds to an antibody that recognizes the RBD of SARS-CoV-2. The identity of the amino acid sequence can be determined by sequence alignment. Sequence alignment itself is known, and can be performed by, for example, a known multiple alignment program such as ClustalW or TREBMAL, commercially available software for protein analysis such as Discovery Studio 2020, or the like.

In the amino acid sequence of the protein (ii), the amino acid sequence of the first region is preferably the same as the amino acid sequence set forth in SEQ ID NO: 1 or 2. That is, when the amino acid sequence of the protein (ii) is compared with the amino acid sequence set forth in SEQ ID NO: 1 or 2 by sequence alignment, it is preferable that the types and positions of the amino acid residues included in the amino acid sequence of the first region coincide between the two.

When the amino acid sequence set forth in SEQ ID NO: 1 and the amino acid sequence set forth in SEQ ID NO: 2 are compared by sequence alignment, the amino acid sequences of the first region coincide with each other, and the amino acid sequence identity of the second region is about 70%. For example, when the amino acid residue in the second region is modified in the amino acid sequence set forth in SEQ ID NO: 1 to design the amino acid sequence of the protein (ii), the second region of the amino acid sequence set forth in SEQ ID NO: 2 may be referenced to determine how to modify the amino acid residue. Alternatively, when the amino acid residue in the second region is modified in the amino acid sequence set forth in SEQ ID NO: 2 to design the amino acid sequence of the protein (ii), the second region of the amino acid sequence set forth in SEQ ID NO: 1 may be referenced to determine how to modify the amino acid residue. The modification of the amino acid residue may be any of substitution, addition, and deletion, but is preferably substitution.

The binding between the chimeric protein of the present embodiment which is the protein (ii) and the antibody that recognizes the RBD of SARS-CoV-2 can be routinely confirmed by a known immunological measurement method. The antibody that recognizes the RBD of SARS-CoV-2 is not particularly limited, and a commercially available anti-SARS-CoV-2 antibody capable of binding to the RBD may be used. Alternatively, monoclonal antibodies represented by the clone names CV30, REGNI10933, BD23, REGNI10987, and CR3022 used in Experimental Example 1 described later may be used. Examples of the immunological measurement method include enzyme-linked immunosorbent assay (ELISA) and immunoprecipitation.

The chimeric protein of the present embodiment may contain an additional oligopeptide or a polypeptide as necessary. Examples of such an oligopeptide and a polypeptide include a signal peptide, a peptide tag, and the like. The peptide tag can be appropriately selected from known peptide tags such as a histidine tag, a glutathione-S-transferase (GST) tag, and a FLAG (registered trademark) tag.

The subject is not particularly limited, but is preferably a subject in which the anti-SARS-CoV-2 antibody is generated or may be generated. Examples of such a subject include a person infected with SARS-CoV-2, a person who has developed COVID-19, a person who has received COVID-19 vaccine, and the like. Examples of the person who has developed COVID-19 include patients with symptoms of COVID-19, asymptomatic patients, and those who have been cured of COVID-19.

The specimen is not particularly limited, but a specimen including or possibly including an anti-SARS-CoV-2 antibody is preferable. Examples of such a specimen include a blood specimen, saliva, nasal mucus, nasopharyngeal swabs, sputum, bronchoalveolar lavage fluid, lymph fluid, tissue fluid, cerebrospinal fluid, and the like. Examples of the blood specimen include blood (whole blood), plasma, serum, and the like. Among them, a blood specimen is preferable, and serum and plasma are particularly preferable.

When insoluble contaminants such as a cell are included in the specimen, the contaminants may be removed from the specimen by known means such as centrifugation or filtration. The specimen may be diluted with an appropriate aqueous medium as necessary. Examples of the aqueous medium include water, physiological saline, and a buffer. The buffer preferably has a buffering action at a pH near neutrality (for example, a pH of 6 or more and 8 or less). Examples of such a buffer include phosphate buffered saline (PBS), Tris-HCl buffer (Tris-HCl), Tris-buffered saline (TBS), Good's buffer, and the like. Examples of the Good's buffer include HEPES, MES, and PIPES.

The detection method of the present embodiment preferably includes contacting a specimen collected from a subject with the chimeric protein of the present embodiment. When the specimen includes an antibody capable of binding to a chimeric protein (that is, an antibody against a chimeric protein), the antibody and the chimeric protein are bound by contact between the specimen and the chimeric protein to form a complex. By detecting this complex, an antibody against the chimeric protein in the specimen can be detected. As described above, it has been confirmed that the chimeric protein of the present embodiment binds to a predetermined anti-SARS-CoV-2 antibody. Therefore, it can be said that the antibody against the detected chimeric protein is an antibody that binds to the RBD of SARS-CoV-2, similarly to a predetermined anti-SARS-CoV-2 antibody. Hereinafter, the antibody against the chimeric protein of the present embodiment in the specimen is also referred to as a “target antibody”.

The method for detecting the complex of the chimeric protein and the target antibody of the present embodiment can be appropriately selected from, for example, known immunological measurement methods. Examples of the immunological measurement method include ELISA, enzyme immunoassay, immunoturbidimetry, immunonephelometry, nephelometric immunoassay, and latex agglutination. Among them, ELISA is preferable. The type of ELISA may be any of a direct method, an indirect method, a sandwich method, a competitive method, and the like. As an example, a case where a target antibody is detected by indirect ELISA is described below.

In the detection of the target antibody by indirect ELISA, a capture body (hereinafter, also referred to as “labeled capture body”) that binds to the target antibody and has a labeling substance is used. The labeled capture body is a substance capable of detecting a complex of the chimeric protein and the target antibody via a signal generated by the labeling substance. The target antibody is a human antibody derived from a subject. Examples of the type of the human antibody include IgG, IgA, and IgM. Therefore, the labeled capture body can be produced by modifying a substance capable of binding to such a human antibody with a labeling substance. Examples of the substance capable of binding to a human antibody include an anti-human IgG antibody, an anti-human IgA antibody, an anti-human IgM antibody, an aptamer, and the like. Details of the labeling substance is described later.

In the detection of the target antibody by indirect ELISA, the following step is performed using the labeled capture body. First, forming an immune complex of the chimeric protein of the present embodiment, the target antibody, and the labeled capture body is performed. Next, detecting a signal generated by the labeling substance included in the immune complex is performed. Each step is described below. In the description, it is assumed that the specimen includes a target antibody.

In the forming an immune complex, the specimen, the chimeric protein, and the labeled capture body are mixed. The order of mixing is not particularly limited, and the specimen, the chimeric protein, and the labeled capture body may be mixed substantially simultaneously or sequentially. Alternatively, after mixing the specimen and the chimeric protein, the labeled capture body may be added to and mixed with the mixture. By mixing, the target antibody in the specimen and the chimeric protein come into contact with each other and bind to each other. As a result, a complex of the chimeric protein and the target antibody is formed. Then, the complex and the labeled capture body come into contact with each other and bind to each other, thereby an immune complex is formed.

In a preferred embodiment, the immune complex is formed on a solid phase on which the chimeric protein of the present embodiment can be immobilized (hereinafter, also simply referred to as “solid phase”). For example, a mixture of the specimen, the chimeric protein, and the labeled capture body is mixed with the solid phase. As a result, the immune complex in the mixture and the solid phase come into contact with each other, and the chimeric protein in the immune complex is immobilized on the solid phase. As a result, an immune complex is formed on the solid phase. Alternatively, a solid phase on which the chimeric protein of the present embodiment is immobilized in advance may be used. By mixing the specimen, the solid phase on which the chimeric protein is immobilized, and the labeled capture body, the target antibody in the specimen, the chimeric protein, and the labeled capture body come into contact with each other on the solid phase, and the immune complex is formed on the solid phase.

The solid phase may be an insoluble carrier capable of immobilizing the chimeric protein of the present embodiment. For example, the chimeric protein can be immobilized on the solid phase by directly or indirectly binding the solid phase to the chimeric protein. Examples of the direct binding between the solid phase and the chimeric protein include adsorption or covalent binding to the surface of the solid phase by hydrophobic interaction. For example, when the solid phase is an ELISA microplate, the chimeric protein can be immobilized in a well of the plate by adsorption. When the solid phase has a functional group on the surface, the chimeric protein can be immobilized on the surface of the solid phase by covalent binding using the functional group. For example, when the solid phase is a particle having a carboxyl group, the crosslink reaction of the compound having a carboxyl group described above can be used. Specifically, a carboxyl group on the particle surface is activated with WSC and then reacted with NHS to form an NHS ester. Then, when the particle having the NHS ester is brought into contact with the chimeric protein, the NHS ester reacts with the amino group of the chimeric protein, and the chimeric protein is immobilized on the particle surface by covalent binding.

Examples of the indirect binding between the solid phase and the chimeric protein include binding via a molecule that specifically binds to the chimeric protein. By previously immobilizing such molecules on the surface of the solid phase, the chimeric protein can be immobilized on the solid phase. Examples thereof include an antibody that specifically recognizes a chimeric protein. In addition, the chimeric protein and the solid phase can be bound to each other by using a combination of a substance mediated between the chimeric protein and the solid phase. Examples of the combination of such a substance include combinations of biotins and avidins, a hapten and an anti-hapten antibody, and the like. The biotins include biotin and biotin analogs such as desthiobiotin and oxybiotin. The avidins include avidin and avidin analogs such as streptavidin and tamavidin (registered trademark). Examples of the combination of a hapten and an anti-hapten antibody include a combination of a compound having a 2,4-dinitrophenyl (DNP) group and an anti-DNP antibody. For example, when the chimeric protein is modified with biotins and avidins are previously immobilized on the solid phase, the chimeric protein can be immobilized on the solid phase via the binding between the biotins and the avidins.

The material of the solid phase is not particularly limited, and for example, can be selected from an organic polymer compound, an inorganic compound, and a biopolymer. Examples of the organic polymer compound include latex, a polystyrene, and a polypropylene. Examples of the inorganic compound include magnetic a substance (iron oxide, chromium oxide, ferrite, and the like), silica, alumina, and glass. Examples of the biopolymer include an insoluble agarose, an insoluble dextran, gelatin, and a cellulose. Two or more kind of these may be used in combination. The shape of the solid phase is not particularly limited, and examples of the shape include a particle, a membrane, a microplate, a microtube, and a test tube. Among them, a microplate and a particle are preferable. The particle is particularly preferably a magnetic particle.

In the detecting a signal, the signal to be detected is generated by the labeling substance of the labeled capture body in the immune complex. The signal generated by the labeling substance reflects the presence or amount of the target antibody in the specimen. That is, the value based on the signal is an index of the presence of the target antibody. In the present specification, “detecting a signal” includes qualitatively detecting the presence or absence of a signal, quantifying the intensity of a signal, and semi-quantitatively detecting the intensity of a signal. “Semi-quantitatively detecting the intensity of a signal” means that the signal intensity is detected in a plurality of stages such as “no signal generation”, “weak”, and “strong”. Preferably, the intensity of the signal generated by the labeling substance included in the immune complex is quantified to obtain a measured value. If necessary, a value obtained by subtracting the background value from the measured value of the signal intensity may be acquired. Examples of the background value include a measured value of signal intensity obtained by measurement without using any one of a specimen, a chimerie protein, and a labeled capture body.

The labeling substance is not particularly limited, and may be, for example, a substance that generates a signal by itself (hereinafter, also referred to as a “signal generating substance”) or may be a substance that catalyzes a reaction of another substance to generate a signal. Examples of the signal generating substance include a fluorescent substance and a radioactive isotope. Examples of the substance that catalyzes a reaction of another substance to generate a detectable signal include an enzyme. Examples of the enzyme include alkaline phosphatase (ALP), peroxidase, ß-galactosidase, and luciferase. Examples of the fluorescent substance include fluorescent dyes such as fluorescein isothiocyanate (FITC), rhodamine, and Alexa Fluor (registered trademark), and fluorescent proteins such as GFP. Examples of the radioactive isotope include 1251, 14° C., and 32P. As the labeling substance, an enzyme is preferable, and ALP is particularly preferable.

The method itself for detecting the signal is known. An appropriate measurement method can be selected according to the type of signal derived from the labeling substance. For example, when the labeling substance is an enzyme, the measurement can be carried out by measuring signals such as light and color generated by reaction between the enzyme and a substrate for the enzyme using a known device. Examples of such a measuring device include a spectrophotometer and a luminometer.

The substrate of the enzyme can be appropriately selected from a known substrate according to a kind of the enzyme. When ALP is used as the enzyme, examples of the substrate include chemiluminescence substrates such as CDP-Star (registered trademark) (disodium 4-chloro-3-(methoxyspiro[1,2-dioxetane-3,2′-(5′-chloro)trixhiro][3.3.1.13,7]decane]-4-yl)phenyl phosphate) and CSPD (registered trademark) (disodium 3-(4-methoxyspiro[1,2-dioxetane-3,2-(5′-chloro)tricyclo[3.3.1.13,7]decane]-4-yl)phenyl phosphate), and coloring substrates such as 5-bromo-4-chloro-3-indrill phosphate (BCIP), disodium 5-bromo-6-chloro-indrill phosphate, and p-nitrophenyl phosphate. When peroxidase is used as the enzyme, examples of the substrate include chemiluminescence substrates such as luminol and its derivative, and coloring substrates such as 2,2′-azinobis(3-ethylbenzothiazolin-6-ammonium sulfonate) (ABTS), 1,2-phenylenediamine (OPD), and 3,3′,5,5′-tetramethylbenzidine (TMB).

When the labeling substance is the radioactive isotope, a radiation as a signal can be measured using a known device such as a scintillation counter. When the labeling substance is the fluorescent substance, fluorescence as a signal can be measured using a known device such as a fluorescent microplate reader. The excitation wavelength and the fluorescence wavelength can be appropriately determined according to a kind of the fluorescent substance used.

The detection result of the signal can be used as a measurement result of the target antibody. For example, when quantitatively detecting the signal intensity, the measured value of the signal intensity itself or a value obtained from the measured value can be used as the measured value of the target antibody. Examples of the value obtained from the measured value of the signal intensity include a value obtained by subtracting a measured value of the negative control sample or a background value from the measured value. The negative control sample can be appropriately selected, and examples thereof include a buffer containing no target antibody, a specimen obtained from a healthy person who has not been infected with SARS-CoV-2, and the like.

Between forming an immune complex and detecting a signal, B/F (Bound/Free) separation for removing an unreacted free component that do not form the complex may be performed. The unreacted free component refers to a component that does not form the immune complex. Examples thereof include the chimeric protein of the present embodiment that did not bond to the target antibody and the labeled capture body. The means for the B/F separation is not particularly limited, when the solid phase is a particle, B/F separation can be performed by recovering only the solid phase in which the immune complex is captured by centrifugation. When the solid phase is a container such as the microplate or microtube, the B/F separation can be performed by removing the liquid containing the unreacted free component. When the solid phase is the magnetic particle, the B/F separation can be performed by suctioning and removing the liquid containing the unreacted free component with a nozzle while the magnetic particle is magnetically constrained by a magnet. The B/F separation is preferable from the viewpoint of automation of measurement. After removing the unreacted free component, the solid phase capturing the immune complex may be washed with a suitable aqueous medium such as PBS.

A further embodiment relates to a polynucleotide encoding the chimeric protein of the present embodiment (hereinafter, also referred to as “polynucleotide of the present embodiment”). The polynucleotide of the present embodiment may be either DNA or RNA. DNA is a stable substance compared to RNA, and various DNA vectors are commercially available. Therefore, the polynucleotide of the present embodiment which is DNA is advantageous in that it is easy to store and handle. It is known that when RNA encoding a protein is introduced into a cell, the protein can be expressed without being affected by a transcriptional regulatory process. Thus, the polynucleotide of the present embodiment, which is RNA, is advantageous in that it enables rapid expression of the chimeric protein in a host cell.

The polynucleotide of the present embodiment may contain various nucleotide sequences in addition to the nucleotide sequence encoding the chimeric protein as necessary. Examples of such a nucleotide sequence include a nucleotide sequence encoding a signal peptide, a recognition sequence of a restriction enzyme, a nucleotide sequence encoding a peptide tag, and a termination codon. The peptide tag can be appropriately selected from known peptide tags such as a histidine tag, a GST tag, and a FLAG (registered trademark) tag.

A further embodiment relates to an expression vector (hereinafter, also referred to as “expression vector of the present embodiment”) including the polynucleotide of the present embodiment. The expression vector of the present embodiment may be in a form in which the polynucleotide of the present embodiment is incorporated into a known vector. The type of vector is not particularly limited as long as it enables protein expression in a host cell. Examples of the type of vector include a plasmid vector and a viral vector. The vector may be linear or circular. The type of plasmid vector is not particularly limited, and examples thereof include an expression vector, a vector for producing a viral vector, and a transposon vector. An expression vector is a vector that allows a protein encoded by a polynucleotide incorporated into the vector to be expressed in an appropriate host cell such as a mammalian cell, an insect cell, a yeast, or an E. coli. The transposon vector is introduced into an appropriate host together with the expression vector into which the gene encoding the transposase is incorporated, thereby allowing the polynucleotide incorporated into the transposon vector to be incorporated into the genome of the host cell.

The type of viral vector is not particularly limited, and examples thereof include a retroviral vector, a lentiviral vector, an adenoviral vector, an adeno-associated virus (AAV) vector, a vaccinia virus vector, and an Epstein-Barr virus (EBV) vector. The viral vector is preferably defective in replication capacity so that the virus does not self-replicate in the infected cell.

The expression vector of the present embodiment may contain an appropriate control sequence as necessary. Examples of such a control sequence include a promoter sequence, an operator sequence, an enhancer sequence, a nucleotide sequence encoding a drug resistance marker, a multiple cloning site, and the like.

A further embodiment relates to a host cell into which the polynucleotide of the present embodiment or the expression vector of the present embodiment has been introduced (hereinafter, also referred to as “host cell of the present embodiment”). The host cell used for introducing the polynucleotide or the expression vector is not particularly limited as long as it can be used as a protein expression system by genetic recombination. Examples of the host cell include a mammalian cell, an insect cell, a yeast, and an E. coli. By introducing a polynucleotide encoding the chimeric protein of the present embodiment or an expression vector including the polynucleotide into a host cell, a host cell including the polynucleotide or the expression vector can be obtained. The method itself for introducing a polynucleotide or an expression vector is known. Examples of such a method include a lipofection method, a calcium phosphate method, and an electroporation method. A commercially available transfection kit may be used. When a viral vector is used as the expression vector, introduction by viral infection can be performed.

A further embodiment relates to a method for producing the chimeric protein of the present embodiment (hereinafter, also referred to as “production method of the present embodiment”). The chimerie protein of the present embodiment can be produced by a known method such as a DNA recombination technique and other molecular biological techniques. For example, first, the polynucleotide of the present embodiment is synthesized. The method for synthesizing a polynucleotide itself is known, and a gene synthesis contract service may be used. When the polynucleotide is DNA, the polynucleotide is incorporated into a known expression vector to obtain the expression vector of the present embodiment. The obtained expression vector is introduced into an appropriate host cell. The details of the host cell are as described above. This results in expression of the chimeric protein in the host cell. When the polynucleotide is RNA, the polynucleotide can be introduced into a host cell. Thereby, the protein can be expressed without being affected by the transcriptional regulatory process. The details of the polynucleotide, the expression vector, and introduction of these are as described above.

In the production method of the present embodiment, a host cell into which a polynucleotide or an expression vector is introduced is cultured for a predetermined period. The host cell can be cultured in the same manner as before a polynucleotide or expression vector is introduced. A culture medium, serum, an additive, and the like can be appropriately determined according to the type of host cell. Examples of the culture medium include MEM, DMEM, and RPMI-1640. Examples of the serum include a fetal bovine serum (FBS) and a human type AB serum. Examples of the additive include L-glutamine and an antibiotic. Examples of the culture conditions for an immune cell include conditions under a 5% CO₂ atmosphere at 37° C.

The predetermined period can be at least a period until the chimeric protein is expressed in the immune cell. The period until the expression of the chimeric protein is determined depending on the method for introducing a polynucleotide or an expression vector, and is, for example, 3 hours or more and 72 hours or less, preferably 6 hours or more and 48 hours or less. When the method for introducing a polynucleotide or an expression vector is a method for enabling stable expression of a chimeric protein in a host cell, the predetermined period of time may be a period of time sufficient for the host cell expressing the chimeric protein to proliferate. Such a period is not particularly limited, but is, for example, 48 hours or more and 20 days or less, preferably 72 hours or more and 14 days or less.

In the production method of the present embodiment, the chimeric protein expressed by the host cell is recovered. When the chimeric protein is produced in a host cell, the host cell is lysed with a solution containing a suitable solubilizing agent. As a result, the chimeric protein is released into the solution. If the host cell secretes the expressed chimeric protein, the chimeric protein is included in the culture supernatant. When insoluble contaminants such as a cell are included in a solubilizing solution or a culture medium containing a chimeric protein, the contaminants may be removed by known means such as centrifugation or filtration. The chimeric protein released in the solubilizing solution or the culture medium can be recovered by a known method such as column chromatography. For example, when the produced chimeric protein has a histidine tag or a GST tag as a peptide tag, it can be recovered by affinity chromatography using a carrier containing Ni-NTA (nitrilotriacetic acid that forms a chelate with a nickel ion) or glutathione. If necessary, the recovered chimeric protein may be purified by a known method such as gel filtration or dialysis.

A further embodiment relates to a reagent for detecting an anti-SARS-CoV-2 antibody containing the chimeric protein of the present embodiment (hereinafter, also referred to as a “reagent of the present embodiment”). The form of the reagent is not particularly limited, and may be, for example, a solid (for example, powder, a crystal, and a lyophilized product) or a liquid (for example, solution, suspension, and emulsion). When the reagent includes a solution of the chimeric protein, the solvent is not particularly limited as long as the chimeric protein of the present embodiment can be dissolved and stored. Examples of the solvent include water, physiological saline, PBS, Tris-HCl, TBS, Good's buffer, and the like. Examples of the Good's buffer include MES, Bis-Tris, ADA, PIPES, Bis-Tris-Propane, ACES, MOPS, MOPSO, BES, TES, HEPES, HEPPS, Tricine, Bicine, TAPS, and the like.

The reagent of the present embodiment may contain a known additive. Examples of the additive include protein stabilizers such as bovine serum albumin (BSA) and sodium caseinate, preservatives such as sodium azide, and inorganic salts such as sodium chloride.

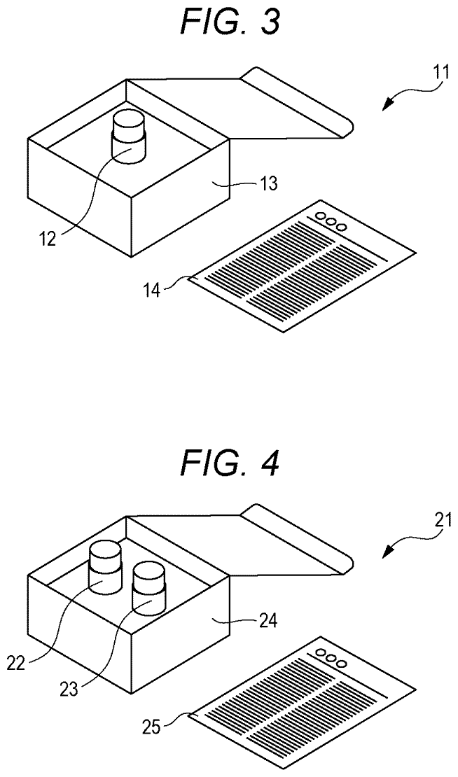

The reagent of the present embodiment may be stored in an appropriate container. In addition, a container including the reagent of the present embodiment may be packed in a box or the like and provided to the user as a reagent kit for detecting an anti-SARS-CoV-2 antibody. The box may include a package insert. The package insert may describe the composition, the method of use, the method of storage, and the like of the reagent of the present embodiment. An example of the reagent kit is shown in FIG. 3. In FIG. 3, 11 denotes a reagent kit for detecting an anti-SARS-CoV-2 antibody, 12 denotes a first container storing the reagent of the present embodiment, 13 denotes a packing box, and 14 denotes a package insert.

The reagent kit may further include a container including a labeled capture body. That is, there is provided a reagent kit for detecting an anti-SARS-CoV-2 antibody, including a first reagent containing the chimeric protein of the present embodiment and a second reagent containing a capture body that binds to an antibody against the chimeric protein and has a labeling substance. An example of the reagent kit is shown in FIG. 4. In FIG. 4, 21 denotes a reagent kit for detecting an anti-SARS-CoV-2 antibody, 22 denotes a first container including a first reagent containing the chimeric protein of the present embodiment, 23 denotes a second container including a second reagent containing a labeled capture body, 24 denotes a packing box, and 25 denotes a package insert. Details of the labeled capture body are as described above.

Hereinafter, the present invention is described in detail with reference to Examples, but the present invention is not limited to these Examples.

### EXAMPLES

### Experimental Example 1: Production of Chimeric Protein and Confirmation of Reactivity (1)

### (1) Design of Chimeric Protein

The Information on the amino acid sequence and structure of a SARS-CoV-2 spike protein was obtained from GenBank and Uniprot (GenBank Accession No. NC_045512.2, Uniprot ID: PODTC2). Based on these information, the amino acid residues of the SARS-CoV-2 spike proteins were numbered and the RBD was defined. Specifically, the amino acid sequence consisting of amino acid residues 319 to 541 of the spike protein shown in SEQ ID NO: 3 (SEQ ID NO: 5) was defined as the RBD of SARS-CoV-2.

As coronaviruses other than SARS-CoV-2, an HKU9 strain and a GCCDC1 strain of Nobecovirus subgenus were selected. For comparison, a Zaria strain and an Hp strain of Hibecovirus subgenus were also selected. The amino acid sequences of the spiked proteins of these strains were obtained from GenBank. For the spike proteins of each strain, Table 1 shows GenBank accession numbers and sequence numbers of the amino acid sequence. In the amino acid sequences of the spike proteins of the respective strains and SARS-CoV-2, sequence alignment was performed using command “Align Sequences (DSC)” in Discovery Studio 2020 (Dassault Systems), and the RBD of each strain was defined.

TABLE 1

| | | GenBank | SEQ ID |
|---|---|---|---|
| Subgenus | Strain | Accession No. | NO: |
| Nobecovirus | HKU9 | YP_001039971.1 | 6 |
| | GCCDC1 | AOG30812.1 | 7 |
| Hibecovirus | Zaria | ADY17911.1 | 8 |
| | Hp | AIL94216.1 | 9 |

From the data registered in the PDB, the structural information of the cocrystal of ACE2 and SARS-CoV-2's RBD was extracted (PDB ID: 6M0J). The amino acid residues interacting with ACE2 and the anti-RBD antibody were identified in the RBD of SARS-CoV-2 using the command “Analyze Protein Interface” in Discovery Studio 2020. These amino acid residues were amino acid residues at positions 346, 372 to 378, 403 to 409, 415, 417, 420, 421 and 435 to 509 of the RBD of SARS-CoV-2 (hereinafter, also referred to as “amino acid residue of first region”). Based on the sequence alignment, the amino acid residues corresponding to the amino acid residues in the first region were identified in the RBDs of the HKU9 strain, the GCCDC1 strain, the Zaria strain, and the Hp strain. The amino acid sequence in which the amino acid residue identified in the RBD of each strain was substituted with the amino acid residue in the first region was designed as the amino acid sequence of the chimeric protein. Table 2 shows the amino acid sequences of the respective chimeric proteins and the viruses from which the first and second regions were derived. In each amino acid sequence in Table 2, the underlined portion indicates the first region, and the remaining sequence indicates the second region.

TABLE 2

| Chimeric | First | Second | |
|---|---|---|---|
| protein | region | region | Amino acid sequence |
| Example 1 | SARS- | HKU9 | RAQVAGFVRVTQRGSYCTPPYSVLQDPRPQPVVWRRYMLYDCVFD |
| | CoV-2 | | FTVVVDSASFSTFKCYGVSPRRLASMCYGSVTLDVMRIRGDEVRQLF |
| | | | NRVTGKFSDYNYALPQNFYGCLHAWNSNNLDSKVGGVYNYLYRLER |
| | | | KSNLKPFERDISTEIYQAGSTPCNGVEGFNCYFPLQSYGFQPTNGVG |
| | | | YQPYRLAVITLKPAAGSKLVCPVANDTVVITDR (SEQ ID NO: 1) |
| Example 2 | | GCCDC1 | RAEAKKLVQVTQQEGSCAIPYTTILEPRPSPAAWVRATISNCTFDFES |
| | | | LLRTASFSTFKCYGISPARLSTMCYAGVTLDIFKLRGDEVRQMLGSVT |
| | | | DKVSDYNYALPSNFYGCVHAWNSNNLDSKVGGNYNYLYRLFRKSNL |
| | | | KPFERDISTEIYQAGSTPCNGVEGFNCYFPLQSYGFQPTNGVGYQPY |
| | | | RGVVVIGLTPASGSNLVCPKANDTHVIEGQ (SEQ ID NO: 2) |
| Comparative | | Zaria | RAEPQGHVVRYAADVADFQQSQGAERLLNASRDQIPDPAFWKRHVI |
| Example 1 | | | RNCKFNFSHIMALASFSTFKCYGIDASKLPSTCWNEVYADVERLRGD |
| | | | EVRQFKPSATGKLADYNYKLPSDFLGCTLAWNSNNLDSKVGGNYNY |
| | | | LYRLFRKSNLKPFERDISTEIYQAGSTPCNGVEGFNCYFPLQSYGFQP |
| | | | TNGVGYQPYRGLALTLKPATVASTVCDVTAQQTNLTLDK (SEQ ID |
| | | | NO: 10) |
| Comparative | | Hp | RATPKQHVVITSADVSAECPFQSLINVTRATIPSPAFWRRHYVRNCN |
| Example 2 | | | YDISVFTDNASFSTFKCYGVAPSSLADMCWEEAHIDYMKIRGDEVRQ |
| | | | FKPSGTGKFADYNYKLPSDFMGCTVAWNSNNLDSKVGGNYNYLYRL |
| | | | FRKSNLKPFERDISTEIYQAGSTPCNGVEGFNCYFPLQSYGFQPTNG |
| | | | VGYQPYRAMAITLKPTRTSATVCGYKQKTTPLVLNE (SEQ ID NO: 11) |

### (2) Expression and Purification of Chimeric Protein

Synthesis and cloning of polynucleotides encoding the each amino acid sequence shown in Table 2 were performed by GeneArt (trademark) gene synthesis (Thermo Fisher Scientific) and TurboGENE (trademark) (GENEWIZ) of gene synthesis contract services. For cloning, each polynucleotide was incorporated into pcDNA (trademark) 3.4 to obtain an expression vector. Using the obtained expression vector and Expi293 (trademark) Expression System (Thermo Fisher Scientific), the expression vector was transiently expressed in an Expi293 (trademark) cell. Transient expression of the chimeric protein was performed according to the manufacturer's protocol, except that the cell was cultured at 28° C. after the enhancer was added until the culture supernatant was recovered. The recovery and purification of the chimeric protein from the culture supernatant were performed by immobilized metal affinity chromatography (IMAC) using a liquid chromatography system Akta pure (trademark) 25 (Cytiva) and a HisTrap HP column (Cytiva). The obtained chimeric protein was purified by size exclusion chromatography (SEC) using a Superdex (registered trademark) 200 increase 10/300 GL column (Cytiva). For comparison, a polynucleotide was synthesized and cloned to obtain a protein for the RBD of each virus of SARS-CoV-2, the HKU9 strain, the GCCDC1 strain, the Zaria strain, and the Hp strain (hereinafter, also referred to as “wild-type RBD”) in the same manner.

### (3) Confirmation of Reactivity of Chimeric Protein to Neutralizing Antibody

### (3.1) Production of Neutralizing Antibody

From the data registered in the PDB, the structural information of the monoclonal antibody in which the neutralization activity against SARS-CoV-2 was confirmed and the structure of the cocrystal with the RBD was analyzed was extracted. Epitopes were identified from the obtained structural information to identify five types of monoclonal antibodies that inhibit the binding between the RBD and human ACE2 (hereinafter, also referred to as “neutralizing antibody”). The polynucleotide sequence of each gene of the heavy chain variable region (VH) and the light chain variable region (VL) of each antibody was obtained from SARS-CoV-2 antibody database (http://opig.stats.ox.ac.uk/webapps/covabdab/). The HTP Gene to Antibody service (Genscript) was utilized to product a recombinant protein of human IgG1 (Kappa) with these VH and VL. Table 3 shows the clone names, PDB ID, amino acid sequences of VH and VL of the neutralizing antibodies. In addition, Table 4 shows the amino acid sequences common to the respective neutralizing antibodies (signal peptide, constant regions of heavy chain and light chain).

TABLE 3

| Clone | PDB_ID | VH | VL |
|---|---|---|---|
| CV30 | 6XE1 | EVQLVESGGGLIPGG | EIVLTQSPGTLSLSP |
| | | SLRLSCAASGVIVSS | GERATLSCRASQSVS |
| | | NYMSWVRQAPGKGLE | SSYLAWYQQKPGQAP |
| | | WVSVIYSGGSTYYAD | RLLIYGASSRATGIP |
| | | SVKGRFTISRDNSKN | DRFSGSGSGTDFTLT |
| | | TLYLQMNSLRAEDTA | ISRLEPEDFAVYYCQ |
| | | VYYCARDLDVSGGMD | QYGSSPQTFGQGTKL |
| | | VWGQGTTVTVSS | EIK |
| | | (SEQ ID NO: 12) | (SEQ ID NO: 13) |
| REGN10933 | 6XDG | QVQLVESGGGLVKPG | DIQMTQSPSSLSASV |
| | | GSLRLSCAASGFTFS | GDRYTTTCQASQDIT |
| | | DYYMSWIRQAPGKGL | NYLNWYQQKPGKAPK |
| | | EWVSYITYSGSTIYY | LLIYAASNLETGVPS |
| | | ADSYKGRFTISRDNA | RFSGSGSGTDFTFTI |
| | | KSSLYLQMNSLRAED | SGLQPEDIATYYCQQ |
| | | TAVYYCARDRGTTMV | YDNLPLTFGGGTKVE |
| | | PFDYWGQGTLVTVSS | IK |
| | | | (SEQ ID NO: 15) |
| | | (SEQ ID NO: 14) | |
| BD23 | 7BYR | QVQLVQSGSELKKPG | DIQMTQSPSTLSASV |
| | | ASVKVSCKASGYTFT | GDRVTTTCRASQSIS |
| | | SYAMNWVRQAPGQGL | SWLAWYQQKPGKAPK |
| | | EWMGWINTNTGNPTY | LLIYKASSLESGVPS |
| | | AQGFTGRFVSLDTSV | RFSGSGSGTEFTLTI |
| | | STAYLQISSLKAEDT | SSLQPDDFATYYCQQ |
| | | AVYYCARPQGGSSWY | YNSYPYTFQQGTKLE |
| | | RDYYYGMDVWGQGTT | IK |
| | | VSS | (SEQ ID NO: 17) |
| | | (SEQ ID NO: 16) | |
| REGN10987 | 6XDG | QVQLVESGGGVVQPG | QSALTQPASVSGSPG |
| | | RSLRLSCAASGFTFS | QSITISCTGTSSDVG |
| | | NYAMYWVRQAPGKGL | GYNYVSWYQQHPGKA |
| | | EWVAVISYDGSNKYY | PKLMIYDVSKRPSGV |
| | | ADSVKGRFTISRDNS | SNRFSGSKSGNTASL |
| | | KNTLYLQMNSLRTED | TISGLQSEDEADYYC |
| | | TAVYYCASGSDYGDY | NSLTSISTWVFGGGT |
| | | LLVYWGQGTLVTVSS | KLTVL |
| | | | (SEQ ID NO: 19) |
| | | (SEQ ID NO: 18) | |
| CR3022 | 6YOR | QMQLVQSGTEVKKPG | DIQMTQSPSSLSASV |
| | | ESLKISCKGSGYGFI | GDRVTTTCRASQSIY |
| | | TYWIGWVRQMPGKGL | SALNWYQQKPGKAPK |
| | | EWMGIIYPGDSETRY | LLIYAASALQSGVPS |
| | | SPSFQGQVTISADKS | RFSGSGSGTDFTLTI |
| | | INTAYLQWSSLKASD | SSLQPEDFATYYCQQ |
| | | TAIYYCAGGSGISTP | TDIHPYTFGQGTKVE |
| | | MDVWGQGTTVTVSS | IK |
| | | (SEQ ID NO: 20) | (SEQ ID NO: 21) |

TABLE 4

| | | Light chain |
|---|---|---|
| Signal | Heavy chain | (Human |
| peptide | (Human IgG1) | Ig Kapa, |
| MGWSGIILFL | ASTKGPSVFPLAPSS | RTVAAPSVFIFPPSD |
| VATATGVHS | KSTSGGTAALGGLVK | EQLKSGTASVVCLLN |
| (SEQ ID | DYFPEPVTVSWNSGA | NFYPREAKVQWKVDN |
| NO: 22) | LTSGVHTFPAVLQSS | ALQSGNSQESVTEQD |
| | GLYSLSSVVTVPSSS | SKDSTYSLSSTLTLS |
| | LGTQTYICNVNVKPS | KADYEKHKVYACEVT |
| | NTKVDKKVEPKSCDK | HQGLSSPVTSFNRGE |
| | THTCPPCPAPELLGG | C |
| | PSVLFPPKPKPKDTL | (SEQ ID NO: 24) |
| | MISRTPEVTQVVVDV | |
| | SHEDPEVKFNWYVDG | |
| | VEVHNAKTKPREEQY | |
| | NSTYRVVSVLTVLHQ | |
| | DWLNGKEYKCKVSNK | |
| | ALPAPIEKTISKAKG | |
| | QPREPQVYTLPPSRD | |
| | ELTKNQVSLTCLVKG | |
| | FYPSDIAVEWESNGQ | |
| | PENNYKTTPPVLDSD | |
| | GSFFLYSKLTVDKSR | |
| | WQQQNVFSCSVMHEA | |
| | LHNHYTQKSLSLSPG | |
| | K | |
| | (SEQ ID NO: 23) | |

(3.2) Confirmation of reactivity by ELISA

### (3.2.1) Preparation of Samples and Reagents

Each of the chimeric protein and the wild-type RBD was diluted with PBS (pH 7.4) to prepare an antigen solution (0.25 μg/mL). As a negative control for the chimeric protein, a 1% BSA solution was used. Each neutralizing antibody was diluted with RI buffer (25 mM HEPES, 150 mM NaCl, 1% BSA, 0.5% Na casein, 1% Tween (trademark) 20, pH 7.5) to prepare an antibody solution (1 μg/mL). As a labeled antibody, a fusion protein of ALP (derived from calf small intestine) and an anti-human IgG antibody was used. The labeled antibody was diluted with R3 buffer (150 mM HEPES, 150 mM NaCl, 1 mM MgCl, 0.1 mM ZnCl, 0.5% Na caseinate, pH 7.4) to prepare a labeled antibody solution (1 μg/mL). ACE2-hFc (AC2-H 5257, Acrobiosystems) was used as a positive control for confirming the binding ability of the chimeric protein to ACE2. This was a recombinant protein in which human ACE2 and the Fc region of human IgG1 were fused. ACE2-hFc was diluted with R1 buffer to prepare ACE2-hFc solution (1 μg/mL).

### (3.2.2) ELISA

To each well of a 96 well plate for ELISA, 100 μL each of an antigen solution and a 1% BSA solution was added, and the well plate was allowed to stand at 4° C. overnight. The solution was removed from the well, 350 μL each of R2 buffer (25 mM HEPES, 150 mM NaCl, 1% BSA, pH 7.5) was added to the each well, and the well plate was allowed to stand at 4° C. overnight. The buffer was removed from the well and washed once with R2 buffer. To the each well, 100 μL of the antibody solution and the ACE2-Fc solution were added, and the well plate was allowed to stand at room temperature for 1 hour. The solution was removed from the each well and washed three times with R2 buffer. To the each well, 100 μL of the labeled antibody solution was added, and the well plate was allowed to stand at room temperature for 30 minutes. The solution was removed from the each well and washed five times with R2 buffer. To each well, 100 μL of a solution of CDP-STAR (registered trademark) was added, and the well plate was allowed to stand in a dark place at room temperature for 10 minutes, and then a signal was detected by a plate reader.

### (4) Confirmation of Reactivity of Chimeric Protein to Blood Specimen

As blood specimens, serums of COVID-19 positive subjects (5 subjects) and COVID-19 negative subjects (2 subjects) were purchased from Cambridge bioscience. Each blood specimen was diluted 10 times with RI buffer to obtain a specimen solution. In ELISA, signals were detected in the same manner as in the above (3.2.2) except that the specimen solution (100 μL/well) was added instead of the antibody solution.

### (5) Results

Table 5 shows the signal intensity in ELISA using the neutralizing antibody and the wild-type RBD or the chimerie protein. In addition. Table 6 shows the signal intensity in ELISA using the blood specimens and the wild-type RBDs or the chimeric proteins. In Table 5. “Blank” indicates data when RI buffer was used as a specimen. In Tables 5 and 6, “BSA” represents data of a negative control.

TABLE 5

| | Antigen | | | | | | | | | |
|---|---|---|---|---|---|---|---|---|---|---|
| | SARS-CoV-2 | HKU9 | GCCDC1 | Zaria | Hp | | | | | |
| | Wild-type | Wild-type | Wild-type | Wild-type | Wild-type | | | Comparative | Comparative | |
| | RBD | RBD | RBD | RBD | RBD | Example 1 | Example 2 | Example 1 | Example 2 | BSA |
| ACE2_hFc | 384,034 | 1,113 | 90 | 150 | 320 | 3,749 | 1751 | 533 | 273 | 23 |
| CV30 | 921,289 | 583 | 130 | 100 | 150 | 11,713 | 8935 | 323 | 193 | 73 |
| RE10933 | 1,004,152 | 943 | 20 | 70 | 60 | 11,671 | 11194 | 283 | 163 | 53 |
| BD23 | 542,993 | 803 | 70 | 240 | 70 | 792 | 1123 | 343 | 23 | 13 |
| RE10987 | 1,039,002 | 1,123 | 70 | 110 | 80 | 8,652 | 3378 | 383 | 183 | 43 |
| CR3022 | 1,068,400 | 433 | 100 | 330 | 100 | 13,279 | 2245 | 343 | 153 | 63 |
| Blank | 33 | 803 | 90 | 150 | 50 | 173 | 339 | 243 | 263 | 13 |

TABLE 6

| | Antigen | | | | | | | | | |
|---|---|---|---|---|---|---|---|---|---|---|
| | SARS-CoV-2 | HKU9 | GCCDC1 | Zaria | Hp | | | | | |
| | Wild-type | Wild-type | Wild-type | Wild-type | Wild-type | | | Comparative | Comparative | |
| | RBD | RBD | RBD | RBD | RBD | Example 1 | Example 2 | Example 1 | Example 2 | BSA |
| Positive | 866,848 | 1,440 | 1,128 | 4991 | 4,030 | 24,775 | 13,964 | 4272 | 4,853 | 1170 |
| specimen 1 | | | | | | | | | | |
| Positive | 736,533 | 840 | 1,775 | 4391 | 952 | 15,381 | 5,172 | 1880 | 1,900 | 630 |
| specimen 2 | | | | | | | | | | |
| Positive | 820,309 | 1,890 | 1,064 | 5712 | 3,193 | 24,121 | 11,209 | 3541 | 3,496 | 710 |
| specimen 3 | | | | | | | | | | |
| Positive | 809,247 | 2,000 | 874 | 3931 | 3,007 | 21,613 | 10,388 | 3677 | 3,051 | 960 |
| specimen 4 | | | | | | | | | | |
| Positive | 802,872 | 2,270 | 2,476 | 2830 | 1,739 | 18,276 | 5,190 | 985 | 995 | 610 |
| specimen 5 | | | | | | | | | | |
| Negative | 3,090 | 1,410 | 2,273 | 3270 | 2,732 | 2,180 | 3,420 | 2660 | 3,440 | 690 |
| specimen 1 | | | | | | | | | | |
| Negative | 5,542 | 1,570 | 2,925 | 4921 | 2,191 | 1,850 | 2,350 | 1940 | 1,900 | 590 |
| specimen 2 | | | | | | | | | | |

As can be seen from Table 5, the signal values in the case of using the HKU9 strain, the GCCDC1 strain, the Zaria strain, the wild-type RBD of the Hp strain, and the chimeric protein of Comparative Example 1 or Comparative Example 2 were all low, and it was shown that the chimeric protein hardly reacted with any of ACE2-hFc and the neutralizing antibody. On the other hand, the signal values in the case of using the wild-type RBD of SARS-CoV-2 and the chimeric protein of Example 1 or Example 2 were all high, and it was shown that they remarkably reacted with ACE2-hFc and all the neutralizing antibodies. The results of Comparative Examples 1 and 2 were considered to be caused by the fact that the three-dimensional structure as an RBD was not maintained in the chimeric protein of the first region derived from SARS-CoV-2 and the second region derived from Hibecovirus subgenus. From these results, it was suggested that the chimeric proteins of Examples 1 and 2 can detect an anti-SARS-CoV-2 antibody that binds to a part involved in binding to ACE2.

As can be seen from Table 6, the signal values in the case of using the HKU9 strain, the GCCDC1 strain, the Zaria strain, the wild-type RBD of the Hp strain, and the chimeric protein of Comparative Example 1 or Comparative Example 2 were all low, and it was shown that the chimeric proteins hardly reacted with any of the positive specimens and the negative specimens. On the other hand, when the SARS-CoV-2 wild-type RBD, the chimeric protein of Example 1 or Example 2 was used, the signal values of the positive specimens were all high, and the signal values of the negative specimens were all low. These results suggested that the chimeric proteins of Examples 1 and 2 can be used as an antigen for detecting an antibody against SARS-CoV-2 in a positive specimen.

### Experimental Example 2: Production of Chimeric Protein and Confirmation of Reactivity (2)

### (1) Design of Chimeric Protein

In Experimental Example 2, a chimeric protein with an RBD of SARS-CoV-2 was further produced for comparison with the chimeric protein of Example 1. As coronaviruses other than SARS-CoV-2, a HKU4 strain, a HKU5 strain and Middle East respiratory syndrome coronavirus (MERS-CoV) of Merbecovirus subgenus, a HKU1 strain and mouse hepatitis virus (MHV) of Embecovirus subgenus, and SARS-CoV of Sarbecovirus subgenus were selected. The amino acid sequences of the spiked proteins of these strains were obtained from GenBank. For the spike protein of each strain, the accession number and the sequence number of the amino acid sequence are shown in Table 7. The RBD of each strain was defined in the same manner as in Experimental Example 1.

TABLE 7

| | | GenBank | SEQ ID |
|---|---|---|---|
| Subgenus | Strain | Accession No. | NO: |
| Merbecovirus | HKU4 | A3EX84 | 25 |
| | HKU5 | A3EXD0 | 26 |
| | MERS-CoV | K9NSQ8 | 27 |
| Embecovirus | HKU1 | QSMQD0 | 28 |
| | MHV | P11224 | 29 |
| Sarbecovirus | SARS-CoV | P59594 | 30 |

In the same manner as in Experimental Example 1, amino acid residues corresponding to the first region of the RBD of SARS-CoV-2 were identified in the RBDs of the HKU4 strain, the HKU5 strain, MERS-CoV, HKU1 and MHV. The amino acid sequence in which the amino acid residue identified in the RBD of each strain was substituted with the amino acid residue in the first region was designed as the amino acid sequence of the chimeric protein. The chimeric proteins including these amino acid sequences were designated as Comparative Example 3 to 7, respectively. The RBD of SARS-CoV was substituted with amino acid residues in the region from position 453 to 509 of the RBD of SARS-CoV-2, based on Shang J. et al., Structural basis of receptor recognition by SARS-CoV-2, Nature, vol. 581, pp. 221-224, 2020. A chimeric protein including the amino acid sequence was designated as Comparative Example 8. Table 8 shows the amino acid sequences of the respective chimeric proteins of Comparative Example 3 to 7. In the table, the “second region” refers to a part other than the amino acid residue corresponding to the first region in the RBDs of HKU4 strain, HKU5 strain, MERS-CoV, HKU1 and MHV. In each amino acid sequence, the underlined portion indicates the first region, and the remaining sequence indicates the second region. For Comparative Example 8, see Shang J. et al., Structural basis of receptor recognition by SARS-CoV-2, Nature, vol. 581, pp. 221-224, 2020.

TABLE 8

| Chimeric | First | Second | Amino acid |
|---|---|---|---|
| protein | region | region | sequence |
| Comparative | SARS-CoV-2 | HKU4 | EASATGTMIEQPNAT |
| Example | | | ECDFSPMLTGRAPQV |
| 3 | | | YNFKRLVFSNCNYNL |
| | | | TKLLSLASFSTFKCN |
| | | | GISPDSIARGCYSTL |
| | | | TVDYFAYRGDEVRQI |
| | | | RPGSTGKIPDYNYKQ |
| | | | SFANPTCRVAWNSNN |
| | | | LDSKVGGNYNYLRLF |
| | | | RKSNLKPFERDISTE |
| | | | IYQAGSTPCNGVEGF |
| | | | NCYFPLQSYGFQPTN |
| | | | GVGVGYQPYRSFIIS |
| | | | VQYGYTGTDSVCPML |
| | | | DLGDSLTITNR |
| | | | (SEQ ID NO: 31) |
| Comparative | | HKUS | EASPRGFIEQATTQE |
| Example | | | CDFTPMLTGTRPPPI |
| 4 | | | YNFKRLVFTNCNYNL |
| | | | TKLLSLASFSTFKCH |
| | | | QVSPSSLATGGYSSL |
| | | | TVDYFAYRGDEVRQL |
| | | | QPGSTGKVIDYNYKQ |
| | | | DFSNPTCRVAWNSNN |
| | | | LDSKVGGNYNYLYRL |
| | | | FRKSNLKPFERDIST |
| | | | EIYQAGSTPGNGVEG |
| | | | FNCYFPLQSYGFQPT |
| | | | NGVNYQPYRAFIISV |
| | | | QYGTDTSNSVCPMQA |
| | | | LRNDTSIE |
| | | | (SEQ ID NO: 32) |
| Comparative | | MERS- | EAKPSGSVVEQAEGV |
| Example | | CoV | ECDFSPLLSGRPPQV |
| 5 | | | YNFKRLVFTNCNYNL |
| | | | TKLLSLASFSTFKCS |
| | | | QISPAAIASNCYSSL |
| | | | ILDYFSYRGDEVRQL |
| | | | SVSSTGKISDYNYKQ |
| | | | SFSNPTCLIAWNSNN |
| | | | LDSKVGGNYNYLYRL |
| | | | FRKSNLKPFERDIST |
| | | | EIYQAGSTPCNGVEG |
| | | | FNCYFPLQSYGFQPT |
| | | | NGVGYQPYRGFGITV |
| | | | QYGTDTNSVPKLEFA |
| | | | NDTKIA |
| | | | (SEQ ID NO: 33) |
| Comparative | | HKU1 | TVKPVATVHRRIDLP |
| Example | | | DCDIDKWLNNFRVPS |
| 6 | | | PLNWERKIFSNCNFN |
| | | | LSTLLRLASFSTFKC |
| | | | NNFDESKIYGSCFKS |
| | | | IVLDFAIRGDEVRQL |
| | | | QLGSTGKLQDYNYKI |
| | | | DTTSSSCQLAWNSNN |
| | | | LDSKVGGNYNYLYRL |
| | | | FRKSNLKPFERDIST |
| | | | EIQAGSTPCNGVEGF |
| | | | NCYFPLQSYGFQPTN |
| | | | GVGYQPYRFSNFILN |
| | | | GINSGTTCSNDLLQP |
| | | | NTEVFT |
| | | | (SEQ ID NO: 34) |
| Comparative | | MHV | TVVQPVGVVYRRVAN |
| Example | | | LPACNIEEWLTARRV |
| 7 | | | PPLNWERKTFQNNNL |
| | | | SSLLRYASFSTFKCN |
| | | | NIDASKVYGRCFGSI |
| | | | SV |
| | | | DKFAVRGDEVRQLQL |
| | | | GNTGKLQDYNYKIDT |
| | | | AATSCQLAWNSNNLD |
| | | | SKVGGNYNYLYRLFR |
| | | | KSNLKPFERDISTEI |
| | | | YQAGSTPCNGVEGFN |
| | | | CYFPLQSYGFQPTNG |
| | | | VGYQPYRFANILLNG |
| | | | INSGTTCSTDLQLPN |
| | | | TEVVTGI |
| | | | (SEQ ID NO: 35) |

### (2) Expression and Purification of Chimeric Protein

Synthesis and cloning of polynucleotides encoding the each amino acid sequence shown in Table 8 were performed in the same manner as in Experimental Example 1 to obtain expression vectors. Using the obtained expression vector and Expi293 (trademark) Expression System (Thermo Fisher Scientific), the expression vector was transiently expressed in an Expi293 (trademark) cell in the same manner as in Experimental Example 1. The recovery and purification of the chimeric protein from the culture supernatant were performed by IMAC in the same manner as in Experimental Example 1. However, the chimeric proteins of the HKU1 strain and the HKU4 strain could not be purified and obtained. Chimeric proteins other than the HKU1 strain and the HKU4 strain could be purified. The obtained chimeric protein was purified by SEC in the same manner as in Experimental Example 1. The chimeric proteins derived from the HKU5 strain, MERS-CoV, and MHV all formed dimers. The chimeric protein from SARS-CoV was monomeric.

### (3) Confirmation of Reactivity of Chimeric Protein to Neutralizing Antibody

ELISA was performed in the same manner as in Experimental Example 1 except that the wild-type RBD of SARS-CoV-2, and the chimeric proteins of Example 1 and Comparative Examples 4, 5, 7 and 8 were used as antigens.

### (4) Confirmation of Reactivity of Chimeric Protein to Blood Specimen

As blood specimens, serums of COVID-19 positive subjects (3 subjects) and COVID-19 negative subjects (3 subjects) were purchased from Cambridge bioscience. ELISA was performed in the same manner as in Experimental Example 1 except that the wild-type RBD of SARS-CoV-2 and the chimeric proteins of Example 1 and Comparative Example 8 were used as antigens.

### (5) Results

Table 9 shows the signal intensity in ELISA using a neutralizing antibody and a chimeric protein. In addition, Table 10 shows the signal intensity in ELISA using the blood specimens and the chimeric proteins.

TABLE 9

| | Antigen | | | | | | |
|---|---|---|---|---|---|---|---|
| | SARS-CoV-2 | | Comparative | Comparative | Comparative | Comparative | |
| | Wild-type RBD | Example 1 | Example 4 | Example 5 | Example 7 | Example 8 | BSA |
| ACE2_hFc | 172033 | 46557 | 103 | 53 | 563 | 238364 | 83 |
| CV30 | 1173539 | 455440 | 63 | 93 | 193 | 420111 | 33 |
| REGN10933 | 1166839 | 614882 | 83 | 53 | 183 | 1081236 | 63 |
| BD23 | 194094 | 5075 | 143 | 173 | 233 | 83 | 83 |
| REGN10987 | 1293259 | 350487 | 163 | 143 | 263 | 193 | 63 |
| ACR3 | 1631137 | 881648 | 153 | 73 | 363 | 1418665 | 63 |
| CR3022 | 1235387 | 944822 | 93 | 113 | 243 | 1536634 | 53 |
| blank | 63 | 223 | 113 | 63 | 183 | 53 | 93 |

TABLE 10

| | Antigen | | | |
|---|---|---|---|---|
| | SARS-CoV-2 | Comparative | | |
| | Wild-type RBD | Example 8 | Example 1 | BSA |
| Positive | 1283126 | 888908 | 633308 | 1603 |
| specimen 1 | | | | |
| Positive | 1020253 | 576647 | 396834 | 1493 |
| specimen 2 | | | | |
| Positive | 1313653 | 802452 | 581933 | 1333 |
| specimen 3 | | | | |
| Negative | 9874 | 2807 | 10846 | 1263 |
| specimen 1 | | | | |
| Negative | 7450 | 5178 | 6079 | 2053 |
| specimen 2 | | | | |
| Negative | 3007 | 1326 | 3767 | 1163 |
| specimen 3 | | | | |
| Blank | 106 | 116 | 176 | 43 |

As can be seen from Table 9, the signal values in the case of using the chimeric protein of Comparative Examples 4, 5 or 7 were all low, and it was shown that the chimeric protein hardly reacted with any of ACE2-hFc and the neutralizing antibody. In the fusion protein of the first region derived from SARS-CoV-2 and the second region derived from Embecovirus subgenus or Merbecovirus subgenus, the three-dimensional structure as the RBD was not maintained, which was considered to be the cause. When the chimeric protein of Comparative Example 8 was used, it reacted with neutralizing antibodies other than BD23 and REGN10987. This is considered to be because the three-dimensional structure of the chimeric protein of Comparative Example 8 as an RBD was maintained. However, since the chimeric protein hardly reacted with BD23 and REGN10987, it can be said that the chimeric protein of Comparative Example 8 had insufficient reactivity with the neutralizing antibody. On the other hand, the signal values in the case of using the wild-type RBD of SARS-CoV-2 or the chimeric protein of Example 1 were all high, and it was shown that they remarkably reacted with ACE2-hFc and all the neutralizing antibodies. From these results, it was suggested that the chimeric protein of Examples 1 can detect an anti-SARS-CoV-2 antibody that binds to a part involved in binding to ACE2.

As can be seen from Table 10, when the SARS-CoV-2 wild-type RBD or the chimeric protein of Comparative Example 8 or Example 1 was used, the signal values of the positive specimens were all high and the signal values of the negative specimens were all low. These results suggested that the chimeric protein of Example 1 can be used as an antigen for detecting an antibody against SARS-CoV-2 in a positive specimen.

### Experimental Example 3: Confirmation of Reactivity to Blood Specimen of Vaccinated Subject

### (1) ELISA

As blood specimens, serums of subjects vaccinated with a COVID-19 vaccine manufactured by Moderna (15 subjects) were purchased from Cambridge bioscience. The serums were 45 specimens, and was prepared from blood collected from 15 subjects at 3 points before vaccination, after the first vaccination, and after the second vaccination. ELISA was performed in the same manner as in Experimental Example 1 except that these serums were used as blood specimens, and the wild-type RBD from SARS-CoV-2 and the chimeric protein from Example 1 were used as antigens.

### (2) Results

Table 11 shows the signal intensity in ELISA using the blood specimens and the chimeric proteins.

TABLE 11

| | Number of | SARS-CoV-2 | |
|---|---|---|---|
| Subject No. | vaccinations | Wild-type RBD | Example 1 |
| 1 | 0 | 2951 | 3661 |
| | 1 | 637911 | 530876 |
| | 2 | 713483 | 637217 |
| 2 | 0 | 2559 | 1497 |
| | 1 | 622604 | 506965 |
| | 2 | 734470 | 654800 |
| 3 | 0 | 4178 | 111374 |
| | 1 | 85454 | 142913 |
| | 2 | 626523 | 575609 |
| 4 | 0 | 2049 | 1195 |
| | 1 | 15102 | 5653 |
| | 2 | 606452 | 532746 |
| 5 | 0 | 170056 | 194667 |
| | 1 | 814440 | 767566 |
| | 2 | 788756 | 724363 |
| 6 | 0 | 3119 | 1605 |
| | 1 | 637771 | 553714 |
| | 2 | 826679 | 745016 |
| 7 | 0 | 3529 | 4947 |
| | 1 | 172159 | 94962 |
| | 2 | 845970 | 731860 |
| 8 | 0 | 3771 | 1570 |
| | 1 | 704623 | 530498 |
| | 2 | 851363 | 790034 |
| 9 | 0 | 12787 | 2588 |
| | 1 | 475715 | 429555 |
| | 2 | 685335 | 640447 |
| 10 | 0 | 67485 | 48226 |
| | 1 | 717214 | 620484 |
| | 2 | 721488 | 650259 |
| 11 | 0 | 3670 | 2464 |
| | 1 | 484515 | 404625 |
| | 2 | 686276 | 611570 |
| 12 | 0 | 7629 | 3385 |
| | 1 | 457661 | 309839 |
| | 2 | 682048 | 579518 |
| 13 | 0 | 3449 | 1269 |
| | 1 | 605930 | 514661 |
| | 2 | 771730 | 697213 |
| 14 | 0 | 11324 | 83921 |
| | 1 | 577480 | 507836 |
| | 2 | 790222 | 698299 |
| 15 | 0 | 821148 | 718429 |
| | 1 | 912837 | 848593 |
| | 2 | 876521 | 795912 |

Except for the subject of No. 15, in the case of using the wild-type RBD from SARS-CoV-2 or the chimeric protein from Example 1, the signal values before vaccination were both low, and the signal values after vaccination were both high. For the subject of No. 15, both the wild-type RBD of SARS-CoV-2 and the chimeric protein of Example 1 were used, and the signal values were high regardless of before and after vaccination. It was considered that the subject of No. 15 was infected with SARS-CoV-2 before vaccination. From the above, it was suggested that the chimeric protein of Example 1 can be used as an antigen for detecting the anti-SARS-CoV-2 antibody induced by vaccination.

What is claimed is:

1. A method for detecting an anti-SARS-CoV-2 antibody in a specimen obtained from a subject, the method comprising:
detecting an antibody against a chimeric protein in the specimen by using the chimeric protein,
the chimeric protein comprising an amino acid sequence of a receptor binding region of SARS-CoV-2 and an amino acid sequence of a receptor binding region of a coronavirus belonging to Nobecovirus subgenus,
the chimeric protein being a protein represented by
(i) a protein comprising an amino acid sequence set forth in SEQ ID NO: 1 or 2; or
(ii) a protein comprising an amino acid sequence having 80% or more of identity to the amino acid sequence set forth in SEQ ID NO: 1 or 2 and binding to an antibody that recognizes the receptor binding region of SARS-CoV-2.

2. The detection method according to claim 1, wherein the protein (ii) comprises an amino acid sequence having 90% or more of identity to the amino acid sequence set forth in SEQ ID NO: 1 or 2.

3. The detection method according to claim 1, wherein the protein (ii) comprises an amino acid sequence having 95% or more of identity to the amino acid sequence set forth in SEQ ID NO: 1 or 2.

4. The detection method according to claim 1,
wherein the detecting an antibody against the chimeric protein comprises:
forming an immune complex of the chimeric protein, an antibody against the chimeric protein, and a capture body that binds to the antibody and has a labeling substance; and
detecting a signal generated by the labeling substance contained in the immune complex.

5. The detection method according to claim 4, wherein a value based on the signal is an index of a presence of the antibody against the chimeric protein in the specimen.

6. The detection method according to claim 1, wherein the specimen is serum or plasma.

7. A chimeric protein comprising an amino acid sequence of a receptor binding region of SARS-CoV-2 and an amino acid sequence of a receptor binding region of a coronavirus belonging to Nobecovirus subgenus, and
the chimeric protein being represented by
(i) a protein comprising an amino acid sequence set forth in SEQ ID NO: 1 or 2; or
(ii) a protein comprising an amino acid sequence having 80% or more of identity to the amino acid sequence set forth in SEQ ID NO: 1 or 2 and binding to an antibody that recognizes the receptor binding region of SARS-CoV-2.

8. The chimeric protein according to claim 7, wherein the protein (ii) comprises an amino acid sequence having 90% or more of identity to the amino acid sequence set forth in SEQ ID NO: 1 or 2.

9. The chimeric protein according to claim 7, wherein the protein (ii) comprises an amino acid sequence having 95% or more of identity to the amino acid sequence set forth in SEQ ID NO: 1 or 2.

10. A polynucleotide encoding the chimeric protein of according to claim 7.

11. An expression vector comprising the polynucleotide according to claim 10.

12. A host cell into which the polynucleotide according to claim 10 is introduced.

13. A host cell into which the expression vector according to claim 11 is introduced.

14. A method for producing a chimeric protein, comprising culturing the host cell according to claim 12 and recovering the chimeric protein expressed by the host cell.

15. A method for producing a chimeric protein, comprising culturing the host cell according to claim 13 and recovering the chimeric protein expressed by the host cell.

16. A reagent for detecting an anti-SARS-CoV-2 antibody, comprising the chimeric protein according to claim 7.


SEQUENCE LISTING (35 sequences)

SEQ ID NO: 1 - protein, 218 aa, synthetic construct
RAQVAGFVRVTQRGSYCTPPYSVLQDPRPQPVVWRRYMLYDCVFDFTVVVDSASFSTFKCYGVSPRRLASMCYGSVTLDVMRIRGDEVRQLFNRVTGKFSDYNYALPDNFYGCLHAWNSNNLDSKVGGNYNYLYRLFRKSNLKPFERDISTEIYQAGSTPCNGVEGFNCYFPLQSYGFQPTNGVGYQPYRLAVITLKPAAGSKLVCPVANDTVVITDR

SEQ ID NO: 2 - protein, 219 aa, synthetic construct
RAEAKKLVQVTQQEGSCAIPYTTILEPRPSPAAWVRATISNCTFDFESLLRTASFSTFKCYGISPARLSTMCYAGVTLDIFKLRGDEVRQMLGSVTDKVSDYNYALPSNFYGCVHAWNSNNLDSKVGGNYNYLYRLFRKSNLKPFERDISTEIYQAGSTPCNGVEGFNCYFPLQSYGFQPTNGVGYQPYRGVVVIGLTPASGSNLVCPKANDTHVIEGQ

SEQ ID NO: 3 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 250 aa, Severe acute respiratory syndrome coronavirus 2
CTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTG

SEQ ID NO: 5 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 6 - protein, 1274 aa, Rousettus bat coronavirus HKU9
MLLILVLGVSLAAASRPECFNPRFTLTPLNHTLNYTSIKAKVSNVLLPDPYIAYSGQTLRQNLFMADMSNTILYPVTPPANGANGGFIYNTSIIPVSAGLFVNTWMYRQPASSRAYCQEPFGVAFGDTFENDRIAILIMAPDNLGSWSAVAPRNQTNIYLLVCSNATLCINPGFNRWGPAGSFIAPDALVDHSNSCFVNNTFSVNISTSRISLAFLFKDGDLLIYHSGWLPTSNFEHGFSRGSHPMTYFMSLPVGGNLPRAQFFQSIVRSNAIDKGDGMCTNFDVNLHVAHLINRDLLVSYFNNGSVANAADCADSAAEELYCVTGSFDPPTGVYPLSRYRAQVAGFVRVTQRGSYCTPPYSVLQDPPQPVVWRRYMLYDCVFDFTVVVDSLPTHQLQCYGVSPRRLASMCYGSVTLDVMRINETHLNNLFNRVPDTFSLYNYALPDNFYGCLHAFYLNSTAPYAVANRFPIKPGGRQSNSAFIDTVINAAHYSPFSYVYGLAVITLKPAAGSKLVCPVANDTVVITDRCVQYNLYGYTGTGVLSKNTSLVIPDGKVFTASSTGTIIGVSINSTTYSIMPCVTVPVSVGYHPNFERALLFNGLSCSQRSRAVTEPVSVLWSASATAQDAFDTPSGCVVNVELRNTTIVNTCAMPIGNSLCFINGSIATANADSLPRLQLVNYDPLYDNSTATPMTPVYWVKVPTNFTLSATEEYIQTTAPKITIDCARYLCGDSSRCLNVLLHYGTFCNDINKALSRVSTILDSALLSLVKELSINTRDEVTTFSFDGDYNFTGLMGCLGPNCGATTYRSAFSDLLYDKVRITDPGFMQSYQKCIDSQWGGSIRDLLCTQTYNGIAVLPPIVSPAMQALYTSLLVGAVASSGYTFGITSAGVIPFATQLQFRLNGIGVTTQVLVENQKLIASSFNNALVNIQKGFTETSIALSKMQDVINQHAAQLHTLVVQLGNSFGAISSSINEIFSRLEGLAANAEVDRLINGRMMVLNTYVTQLLIQASEAKAQNALAAQKISECVKAQSLRNDFCGNGTHVLSIPQLAPNGVLFIHYAYTPTEYAFVQTSAGLCHNGTGYAPRQGMFVLPNNTNMWHFTTMQFYNPVNISASNTQVLTSCSVNYTSVNYTVLEPSVPGDYDFQKEFDKFYKNLSTIFNNTFNPNDFNFSTVDVTAQIKSLHDVVNQLNQSFIDLKKLNVYEKTIKWPWYVWLAMIAGIVGLVLAVIMLMCMTNCCSCFKGMCDCRRCCGSYDSYDDVYPAVRVNKKRTV

SEQ ID NO: 7 - protein, 1290 aa, Rousettus bat coronavirus GCCDC1
MFRTLLLAALFNACWCARPSCLKPTEYWGTHTFDGVEYVPHNTTYVSVSLNKLSCLLPDPYMAHSGQTVRQKLYMGNTSNTLVYPVTPPMFNLTYGNVTPGVYNTSFLPVFDGLLVHTYMNRFAHLDNPNRTCQEPFGVVFGTTFEQDRIAMVIIAPGEFGMWGQLTRPNTTYVHVVACSNATVCAYPMFNRWGPAGSIHADDAFVEHNSSCFLNDTFEIPMGTSRVNLAFRFQDGNLLLYHTAWLPTENYTLSGDYPLRYARSVGVGSNLPFAQFFQSVARARDSACAFLHNNLYIAPVQPKELLVKYNDQGMPVEIADCSADSTQELYCITGTFTPSVGVYQLSRYRAEAKKLVQVTQQEGSCAIPYTTILEPPSPAAWVRATISNCTFDFESLLRTLPTYNLKCYGISPARLSTMCYAGVTLDIFKLNTTHLSNMLGSVPDAVSIYNYALPSNFYGCVHAYHLNSTTPYAVAVPPGAYPIKPGGRQLFNSFVSQVLDSPSSQCTPANCMGVVVIGLTPASGSNLVCPKANDTHVIEGQCVKYNFYGYAGTGVINQSDLAIPNNKLFVTSKSGAVLAVRAGDKVYSITPCVSVPISVGYDPGHQRALVFNGLDCSARANAVSMPASEYWTAAASTIARGSEPVLDTPSGCVYNVNNCTTHTVSVCEMPIGNSLCLVSNFTCSNVATANLSPNLLSLVVYDPTDAGLKVLTPVYWVSIPTNFTLAATTEYIQTTAPKVNVDCVKYLCGDSERCIDVLSQYGAFCEDVNKALADVSAIIDSSMVTMVSELTAGVMWSETPQANVGSYNFSGLMGCLGSNCQEKQYRSAISDLLYNKVKVADPGFMGAYQKCIDEQWGGSVRDLICTQTFNGISVLPPIVSPGMQALYTSLLVGAVASSGYTFGITSVGVIPFATQLQFRLNGLGVTTNVLMENQKLIANAFNNALTGIQKGFDATNMALAKMQSVINQHAQQLSTLVDQLGNSFGAISSSINEIFSRLDELEANAQVDRLINGRMVVLNTYVTQLLIRASEVKAQAALASQKISECVKAQSQRNDFCGNGTHVLSIPQMAPNGVLFLHYSYQPTAYNLVRTAAGLCFNDTGYAPLGGLFVLPNNTDRWLFTKMSFYDPVNISVSNTQVLAACGLNYSSVNYTVLEPAVDTSSFNFTEEFEKWYVNQSHIFNNTFNASAFNFSLVDVNEQLAILTDVVNQLNQSYIDLKQLGTYEYTVKWPWYVWLGMIAGLVGLVLAVVLLLCMTNCCSCARGVCSCKSCAYEEHEDVYPAVRVHGKRTA

SEQ ID NO: 8 - protein, 1298 aa, Zaria bat coronavirus
MSFIVAFTVLLPIVTAAPSFAGGCKTYSLSGAYTENNYTINGTVSYGLYYPDAIFRSNVSVLFTRPFLPLHAEAYQWYGAYKDQGVSINYQYSYLPHPFKDGIAVKIFHRVREGYTKGFSTPAAYLFGSTFDSNSSTLLIGVNGTHRIIAVCQFVVCKSPLVHLYPYNQTTPPDTFTKYYLEGLFSAADNCSLLIEQPLWLDNSTNSNRLVTAQTQYIFTYESGVFSVYHAHNPNGSVGYATLAFTIPVTIPITKFYLPAVFDGLKASGDNGVKRGNYHIEFAYLKRTTYMFTYDGNGYITSHVYCAEGPLQELKCSQLSNNVPPGVYRTTNYRAEPQGHVVRYAADVADFQQSCGAERLLNASIDQIPDPAFWKRHVIRNCKFNFSHIMALSHVYDMQCYGIDASKLPSTCWNEVYADVFRLAQDDFYSFKPSASGDLATYNYKLPSDFLGCTLILTNPNLYCGNSTTCGIPGFNGVTGDLHYKVWRPGTTICPMKYVNPGEQNAMGYHCMSPTEHEKKLTPVSFRGGYYYMYGLALTLKPATVASTVCDVTAQQTNLTLDKCVSYTVYGYNGQGILVETNETFPSFQNVQLWPNGQLKAFKDPQSNQVIAVLPCAEADVSVATAGNHTNEVATLFTGAPCSGVQAKLLGVSTTSMWRRISENTTKGIDTPVGCLFGAVLSTSNSTECQFSLGLDTCLNITRGRVGSRSAGHLKESSTYNYFFQDMPKESEIPFSNFTVEIPDNFTIAIEYETLPIRMSKISVDCSRYVCGTDVVCANQLVQYGKFCDNINEALRGVSLQQDSNVAELLSDIKQLTRVSDLDLKDINGFNFSSLYNTDYLDATSSGRRSAIEDLLFNKVVTADVGFMKKYEECTGGSVFKDLDCAQSFNGLKVLPPQMSDAHVGVYTTAAAMGSFFSIPNTMQMAYRFNGIAVTQSVLVDNQKEIANKFNQALTSIQQGFTATNSAVQKLQDVVNANAAALNTLVTQLGNNFGAISSAINDITQRLDKLEAAVQIDRLINGRLQVLQTFVTQQLIMASEIRASAQLAKQKMSECVLSQSKRQDFCGRGLHLMSFPQSAPQGMVFLHVLYRPTSYINVTSTPAICSENKAYFPTDGVFVLHDNQWMITKRNFYDPVNISLSNVRYAGSCDVITTYANHTIFEPDNSTLQDFMHELEEIRKNLSMSAPNSPNFTLPGLSDINASFVDLSKEMETLQNVVRQLNKSVIDLKELGTYEYYEKWPWYIWLGFIAGLVAIALVLVLICCSTSCCSCFKGMCSCKKCCDSYDDDEQILIKQHYP

SEQ ID NO: 9 - protein, 1317 aa, Bat Hp-betacoronavirus Zhejiang2013
MVARYRLCILQLILSACHLSITRDVQPYVHEANCTKQVATEGPFMAAYPLGKYGFINTYYPDRIFRSNTTALFTDLFPVIGTTVTPWKAFNALTTNERKDKRNFERHPFGEGVFVTIKHTQVSGPSTPPMYLFGTYFNSSSYTLAISLDGLYRRIEVCNFQVCQNPVMYYAKVSNPAQGNPLIYSQRAYTDYNSCVSQTSQSFNITSQGDKNGAFSKDTITTYVFKYQNGIFSVFHGFHNGTDHVPYPKPAFEFPLDIPITHFIIPPTVEEPAQGQGYTRGNYVVYYAQLKKSTFMFGFDSNGLITRYIDCAAGPKDELMCSQGSFNITPGVYTTTNYRATPKQHVVITSADVSAECPFQSLINVTEATIPSPAFWRRHYVRNCNYDISVFTDNADVYSLQCYGVAPSSLADMCWEEAHIDYMKISEKDIFSFKPSGAGDFAKYNYKLPSDFMGCTVVFTNQELTCNATSGQLCHVYTNNLTNYPAEATAWDKSHYESIERYQMWSSENVYNCELQEVGPQQFNRMPRNCYVRSNNSNDYWQPMSFRGVNSLMVAMAITLKPTRTSATVCGYKQKTTPLVLNECITYHIYGYKGTGVIVSANYTFQSFQTVQLTSTGSLHSFKYNNTIYGILPCAQAQVSIVTAGSNHDNVAVVYNGLGCNAFQSKMQGVDNTEWSVYTNFSSPIDTPVGCLTGAIQAQENSTECQFALGLDSCVNYTADTRLRTARAADRALTFNYMYFAQELPSQSEQSVAFDNSTLQIPINFTIGIEYETIPVTMPKVTVDCAQYVCGDNSECRTLLVQYGTFCETINDALRGVALQQDVNQKDVFSSVRRLAKVSSPMNLGNLNGFNFSSILNQEAINGNNFQARSAIEDLFFDKIETADVGFQKKYDECTGGSVVKDLGCAQSFNGLMVLPPQMTDAHVAAYTTSAALGSFFSIPNTMQMAYRFNGIAVTQKVLVDNQKQIANKFNQAMMSVQTGFKATSSALEKLQKVVNDNAQALNNLVAQLTNNFGAISSAINDITQRLDKLEADAQIDRLINGRLQVLQTFVTQQLIRASEIRASAKLAAQKMSECVQGQSQRLDFCGRGMHLMSFPQAAPYGMVFLHVLYKPTDYINVTTVPAICSNDVAYFPVDGVFVYYNNSLMFTKRNYFEPEIITIDNIRSAGSCNVDVTYVNHTIYNPEMPELEDFKQELEEIRKNFSTGLPNRPNFTLPGLEGINASMVDLSEEVKILNDVVKELNASLINLQELGVNSQYIKWPWYIWLGFIAGLIAIVMATIMLCCMTSCCSCFKGLCACKRCCDSFDEPDEPIKYHYP

SEQ ID NO: 10 - protein, 226 aa, synthetic construct
RAEPQGHVVRYAADVADFQQSCGAERLLNASRDQIPDPAFWKRHVIRNCKFNFSHIMALASFSTFKCYGIDASKLPSTCWNEVYADVFRLRGDEVRQFKPSATGKLADYNYKLPSDFLGCTLAWNSNNLDSKVGGNYNYLYRLFRKSNLKPFERDISTEIYQAGSTPCNGVEGFNCYFPLQSYGFQPTNGVGYQPYRGLALTLKPATVASTVCDVTAQQTNLTLDK

SEQ ID NO: 11 - protein, 223 aa, synthetic construct
RATPKQHVVITSADVSAECPFQSLINVTRATIPSPAFWRRHYVRNCNYDISVFTDNASFSTFKCYGVAPSSLADMCWEEAHIDYMKIRGDEVRQFKPSGTGKFADYNYKLPSDFMGCTVAWNSNNLDSKVGGNYNYLYRLFRKSNLKPFERDISTEIYQAGSTPCNGVEGFNCYFPLQSYGFQPTNGVGYQPYRAMAITLKPTRTSATVCGYKQKTTPLVLNE

SEQ ID NO: 12 - protein, 118 aa, synthetic construct
EVQLVESGGGLIQPGGSLRLSCAASGVIVSSNYMSWVRQAPGKGLEWVSVIYSGGSTYYADSVKGRFTISRDNSKNTLYLQMNSLRAEDTAVYYCARDLDVSGGMDVWGQGTTVTVSS

SEQ ID NO: 13 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPQTFGQGTKLEIK

SEQ ID NO: 14 - protein, 120 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSS

SEQ ID NO: 15 - protein, 107 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIK

SEQ ID NO: 16 - protein, 126 aa, synthetic construct
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARPQGGSSWYRDYYYGMDVWGQGTTVTVSS

SEQ ID NO: 17 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIK

SEQ ID NO: 18 - protein, 120 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 19 - protein, 110 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 20 - protein, 119 aa, synthetic construct
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSS

SEQ ID NO: 21 - protein, 107 aa, synthetic construct
DIQMTQSPSSLSASVGDRVTITCRASQSIYSALNWYQQKPGKAPKLLIYAASALQSGVPSRFSGSGSGTDFTLTISSLQPEDFATYYCQQTDIHPYTFGQGTKVEIK

SEQ ID NO: 22 - protein, 19 aa, synthetic construct
MGWSCIILFLVATATGVHS

SEQ ID NO: 23 - protein, 330 aa, Homo sapiens [sequence omitted]

SEQ ID NO: 24 - protein, 107 aa, Homo sapiens [sequence omitted]

SEQ ID NO: 25 - protein, 1352 aa, Tylonycteris bat coronavirus HKU4
MTLLMCLLMSLLIFVRGCDSQFVDMSPASNTSECLESQVDAAAFSKLMWPYPIDPSKVDGIIYPLGRTYSNITLAYTGLFPLQGDLGSQYLYSVSHAVGHDGDPTKAYISNYSLLVNDFDNGFVVRIGAAANSTGTIVISPSVNTKIKKAYPAFILGSSLTNTSAGQPLYANYSLTIIPDGCGTVLHAFYCILKPRTVNRCPSGTGYVSYFIYETVHNDCQSTINRNASLNSFKSFFDLVNCTFFNSWDITADETKEWFGITQDTQGVHLYSSRKGDLYGGNMFRFATLPVYEGIKYYTVIPRSFRSKANKREAWAAFYVYKLHQLTYLLDFSVDGYIRRAIDCGHDDLSQLHCSYTSFEVDTGVYSVSSYEASATGTFIEQPNATECDFSPMLTGVAPQVYNFKRLVFSNCNYNLTKLLSLFAVDEFSCNGISPDSIARGCYSTLTVDYFAYPLSMKSYIRPGSAGNIPLYNYKQSFANPTCRVMASVLANVTITKPHAYGYISKCSRLTGANQDVETPLYINPGEYSICRDFSPGGFSEDGQVFKRTLTQFEGGGLLIGVGTRVPMTDNLQMSFIISVQYGTGTDSVCPMLDLGDSLTITNRLGKCVDYSLYGVTGRGVFQNCTAVGVKQQRFVYDSFDNLVGYYSDDGNYYCVRPCVSVPVSVIYDKSTNLHATLFGSVACEHVTTMMSQFSRLTQSNLRRRDSNIPLQTAVGCVIGLSNNSLVVSDCKLPLGQSLCAVPPVSTFRSYSASQFQLAVLNYTSPIVVTPINSSGFTAAIPTNFSFSVTQEYIETSIQKVTVDCKQYVCNGFTRCEKLLVEYGQFCSKINQALHGANLRQDESVYSLYSNIKTTSTQTLEYGLNGDFNLTLLQVPQIGGSSSSYRSAIEDLLFDKVTIADPGYMQGYDDCMKQGPQSARDLICAQYVSGYKVLPPLYDPNMEAAYTSSLLGSIAGAGWTAGLSSFAAIPFAQSMFYRLNGVGITQQVLSENQKLIANKFNQALGAMQTGFTTSNLAFSKVQDAVNANAQALSKLASELSNTFGAISSSISDILARLDTVEQDAQIDRLINGRLISLNAFVSQQLVRSETAARSAQLASDKVNECVKSQSKRNGFCGSGTHIVSFVVNAPNGFYFFHVGYVPTNYTNVTAAYGLCNNNNPPLCIAPIDGYFITNQTTTYSVDTEWYYTGSSFYKPEPITQANSRYVSSDVKFDKLENNLPPPLLENSTDVDFKDELEEFFKNVTSHGPNFAEISKINTTLLDLSDEMAMLQEVVKQLNDSYIDLKELGNYTYYNKWPWYVWLGFIAGLVALLLCVFFLLCCTGCGTSCLGKMKCKNCCDSYEEYDVEKIHVH

SEQ ID NO: 26 - protein, 1352 aa, Pipistrellus bat coronavirus HKU5
MIRSVLVLMCSLTFIGNLTRGQSVDMGHNGTGSCLDSQVQPDYFESVHTTWPMPIDTSKAEGVIYPNGKSYSNITLTYTGLYPKANDLGKQYLFSDGHSAPGRLNNLFVSNYSSQVESFDDGFVVRIGAAANKTGTTVISQSTFKPIKKIYPAFLLGHSVGNYTPSNRTGRYLNHTLVILPDGCGTILHAFYCVLHPRTQQNCAGETNFKSLSLWDTPASDCVSGSYNQEATLGAFKVYFDLINCTFRYNYTITEDENAEWFGITQDTQGVHLYSSRKENVFRNNMFHFATLPVYQKILYYTVIPRSIRSPFNDRKAWAAFYIYKLHPLTYLLNFDVEGYITKAVDCGYDDLAQLQCSYESFEVETGVYSVSSFEASPRGEFIEQATTQECDFTPMLTGTPPPIYNFKRLVFTNCNYNLTKLLSLFQVSEFSCHQVSPSSLATGCYSSLTVDYFAYSTDMSSYLQPGSAGAIVQFNYKQDFSNPTCRVLATVPQNLTTITKPSNYAYLTECYKTSAYGKNYLYNAPGAYTPCLSLASRGFSTKYQSHSDGELTTTGYIYPVTGNLQMAFIISVQYGTDTNSVCPMQALRNDTSIEDKLDVCVEYSLHGITGRGVFHNCTSVGLRNQRFVYDTFDNLVGYHSDNGNYYCVRPCVSVPVSVIYDKASNSHATLFGSVACSHVTTMMSQFSRMTKTNLLARTTPGPLQTTVGCAMGFINSSMVVDECQLPLGQSLCAIPPTTSSRVRRATSGASDVFQIATLNFTSPLTLAPINSTGFVVAVPTNFTFGVTQEFIETTIQKITVDCKQYVCNGFKKCEDLLKEYGQFCSKINQALHGANLRQDESIANLFSSIKTQNTQPLQAGLNGDFNLTMLQIPQVTTGERKYRSTIEDLLFNKVTIADPGYMQGYDECMQQGPQSARDLICAQYVAGYKVLPPLYDPYMEAAYTSSLLGSIAGASWTAGLSSFAAIPFAQSIFYRLNGVGITQQVLSENQKIIANKFNQALGAMQTGFTTTNLAFNKVQDAVNANAMALSKLAAELSNTFGAISSSISDILARLDTVEQEAQIDRLINGRLTSLNAFVAQQLVRTEAAARSAQLAQDKVNECVKSQSKRNGFCGTGTHIVSFAINAPNGLYFFHVGYQPTSHVNATAAYGLCNTENPQKCIAPIDGYFVLNQTTSTVADSDQQWYYTGSSFFHPEPITEANSKYVSMDVKFENLTNRLPPPLLSNSTDLDFKEELEEFFKNVSSQGPNFQEISKINTTLLNLNTELMVLSEVVKQLNESYIDLKELGNYTFYQKWPWYIWLGFIAGLVALALCVFFILCCTGCGTSCLGKLKCNRCCDSYDEYEVEKIHVH

SEQ ID NO: 27 - protein, 1353 aa, Middle East respiratory syndrome-related coronavirus
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 28 - protein, 1356 aa, Human coronavirus HKU1
MLLIIFILPTTLAVIGDFNCTNFAINDLNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGRVSAAFHQNASSLALLYRNLKCSYVLNNISLTTQPYFDSYLGCVFNADNLTDYSVSSCALRMGSGFCVDYNSPSSSSSRRKRRSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDALEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPKQGYFIKHNDHWMFTGSSYYYPEPISDKNVVFMNTCSVNFTKAPLVYLNHSVPKLSDFESELSHWFKNQTSIAPNLTLNLHTINATFLDLYYEMNLIQESIKSLNNSYINLKDIGTYEMYVKWPWYVWLLISFSFIIFLVLLFFICCCTGCGSACFSKCHNCCDEYGGHHDFVIKTSHDD

SEQ ID NO: 29 - protein, 1324 aa, Murine coronavirus
MLFVFILFLPSCLGYIGDFRCIQLVNSNGANVSAPSISTETVEVSQGLGTYYVLDRVYLNATLLLTGYYPVDGSKFRNLALRGTNSVSLSWFQPPYLNQFNDGIFAKVQNLKTSTPSGATAYFPTIVIGSLFGYTSYTVVIEPYNGVIMASVCQYTICQLPYTDCKPNTNGNKLIGFWHTDVKPPICVLKRNFTLNVNADAFYFHFYQHGGTFYAYYADKPSATTFLFSVYIGDILTQYYVLPFICNPTAGSTFAPRYWVTPLVKRQYLFNFNQKGVITSAVDCASSYTSEIKCKTQSMLPSTGVYELSGYTVQPVGVVYRRVANLPACNIEEWLTARSVPSPLNWERKTFQNCNFNLSSLLRYVQAESLFCNNIDASKVYGRCFGSISVDKFAVPRSRQVDLQLGNSGFLQTANYKIDTAATSCQLHYTLPKNNVTINNHNPSSWNRRYGFNDAGVFGKNQHDVVYAQQCFTVRSSYCPCAQPDIVSPCTTQTKPKSAFVNVGDHCEGLGVLEDNCGNADPHKGCICANNSFIGWSHDTCLVNDRCQIFANILLNGINSGTTCSTDLQLPNTEVVTGICVKYDLYGITGQGVFKEVKADYYNSWQTLLYDVNGNLNGFRDLTTNKTYTIRSCYSGRVSAAFHKDAPEPALLYRNINCSYVFSNNISREENPLNYFDSYLGCVVNADNRTDEALPNCDLRMGAGLCVDYSKSRRAHRSVSTGYRLTTFEPYTPMLVNDSVQSVDGLYEMQIPTNFTIGHHEEFIQTRSPKVTIDCAAFVCGDNTACRQQLVEYGSFCVNVNAILNEVNNLLDNMQLQVASALMQGVTISSRLPDGISGPIDDINFSPLLGCIGSTCAEDGNGPSAIRGRSAIEDLLFDKVKLSDVGFVEAYNNCTGGQEVRDLLCVQSFNGIKVLPPVLSESQISGYTTGATAAAMFPPWSAAAGVPFSLSVQYRINGLGVTMNVLSENQKMIASAFNNALGAIQDGFDATNSALGKIQSVVNANAEALNNLLNQLSNRFGAISASLQEILTRLEAVEAKAQIDRLINGRLTALNAYISKQLSDSTLIKVSAAQAIEKVNECVKSQTTRINFCGNGNHILSLVQNAPYGLYFIHFSYVPISFTTANVSPGLCISGDRGLAPKAGYFVQDDGEWKFTGSSYYYPEPITDKNSVIMSSCAVNYTKAPEVFLNTSIPNPPDFKEELDKWFKNQTSIAPDLSLDFEKLNVTLLDLTYEMNRIQDAIKKLNESYINLKEVGTYEMYVKWPWYVWLLIGLAGVAVCVLLFFICCCTGCGSCCFKKCGNCCDEYGGHQDSIVIHNISSHED

SEQ ID NO: 30 - protein, 1255 aa, Severe acute respiratory syndrome-related coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 31 - protein, 219 aa, synthetic construct
EASATGTFIEQPNATECDFSPMLTGRAPQVYNFKRLVFSNCNYNLTKLLSLASFSTFKCNGISPDSIARGCYSTLTVDYFAYRGDEVRQIRPGSTGKIPDYNYKQSFANPTCRVAWNSNNLDSKVGGNYNYLYRLFRKSNLKPFERDISTEIYQAGSTPCNGVEGFNCYFPLQSYGFQPTNGVGYQPYRSFIISVQYGTGTDSVCPMLDLGDSLTITNR

SEQ ID NO: 32 - protein, 218 aa, synthetic construct
EASPRGEFIEQATTQECDFTPMLTGTRPPPIYNFKRLVFTNCNYNLTKLLSLASFSTFKCHQVSPSSLATGCYSSLTVDYFAYRGDEVRQLQPGSTGKIVDYNYKQDFSNPTCRVAWNSNNLDSKVGGNYNYLYRLFRKSNLKPFERDISTEIYQAGSTPCNGVEGFNCYFPLQSYGFQPTNGVGYQPYRAFIISVQYGTDTNSVCPMQALRNDTSIE

SEQ ID NO: 33 - protein, 217 aa, synthetic construct
EAKPSGSVVEQAEGVECDFSPLLSGRPPQVYNFKRLVFTNCNYNLTKLLSLASFSTFKCSQISPAAIASNCYSSLILDYFSYRGDEVRQLSVSSTGKISDYNYKQSFSNPTCLIAWNSNNLDSKVGGNYNYLYRLFRKSNLKPFERDISTEIYQAGSTPCNGVEGFNCYFPLQSYGFQPTNGVGYQPYRGFGITVQYGTDTNSVCPKLEFANDTKIA

SEQ ID NO: 34 - protein, 219 aa, synthetic construct
TVKPVATVHRRIPDLPDCDIDKWLNNFRVPSPLNWERKIFSNCNFNLSTLLRLASFSTFKCNNFDESKIYGSCFKSIVLDKFAIRGDEVRQLQLGSTGKLQDYNYKIDTTSSSCQLAWNSNNLDSKVGGNYNYLYRLFRKSNLKPFERDISTEIYQAGSTPCNGVEGFNCYFPLQSYGFQPTNGVGYQPYRFSNFILNGINSGTTCSNDLLQPNTEVFT

SEQ ID NO: 35 - protein, 221 aa, synthetic construct
TVQPVGVVYRRVANLPACNIEEWLTARRVPSPLNWERKTFQNCNFNLSSLLRYASFSTFKCNNIDASKVYGRCFGSISVDKFAVRGDEVRQLQLGNTGKLQDYNYKIDTAATSCQLAWNSNNLDSKVGGNYNYLYRLFRKSNLKPFERDISTEIYQAGSTPCNGVEGFNCYFPLQSYGFQPTNGVGYQPYRFANILLNGINSGTTCSTDLQLPNTEVVTGI